(19) United States
(12) Patent Application Publication
(10) Pub. No.: US 2025/0115887 A1
(43) Pub. Date: Apr. 10, 2025

(54) HAPLOTYPE-BASED TREATMENT OF RP1 ASSOCIATED RETINAL DEGENERATIONS

(71) Applicant: Massachusetts Eye and Ear Infirmary, Boston, MA (US)

(72) Inventors: Qin Liu, Boston, MA (US); Caitlin Collin, Boston, MA (US)

(21) Appl. No.: 18/890,708

(22) Filed: Sep. 19, 2024

Related U.S. Application Data
(62) Division of application No. 17/175,234, filed on Feb. 12, 2021, now Pat. No. 12,129,496.
(60) Provisional application No. 62/975,636, filed on Feb. 12, 2020.

(51) Int. Cl. C12N 9/22; A61K 9/00; A61K 38/00; A61K 38/16; C12N 15/11; C12N 15/90

(52) U.S. Cl. CPC C12N 9/22; A61K 9/0048; A61K 38/164; C12N 15/11; C12N 15/907; A61K 38/00; C12N 2310/20; C12N 2800/80

(57) ABSTRACT

Methods and compositions for the use of CRISPR/Cas9 technology for treating RP1 mutation-associated autosomal dominant Retinitis Pigmentosa (adRP).

### CROSS-REFERENCE TO RELATED APPLICATIONS

This application is a continuation of U.S. patent application Ser. No. 17/175,234, filed Feb. 12, 2021, which claims priority and benefit from U.S. Provisional Patent Application 62/975,636, filed Feb. 12, 2020. The entire contents and disclosure of the foregoing are incorporated herein by reference.

### SEQUENCE LISTING

This application contains a Sequence Listing that has been submitted electronically as an XML file named “00633-0277002_SL_ST26.XML.” The XML file, created on Sep. 19, 2024, is 130,506 bytes in size. The material in the XML file is hereby incorporated by reference in its entirety.

### TECHNICAL FIELD

Described herein are methods and compositions for the use of CRISPR/Cas9 technology for treating retinitis pigmentosa 1 protein (RP1) mutation-associated autosomal dominant Retinitis Pigmentosa (adRP).

### BACKGROUND

Retinitis pigmentosa (RP) is an important cause of vision loss for people of all ages. As a group, these diseases are characterized by the progressive death of the light sensitive photoreceptor cells of the retina, which are required for vision. RP is highly heterogeneous genetically, and mutations in the RP genes can be passed from parent to offspring in multiple genetic inheritance modes—autosomal recessive, autosomal dominant, or X-linked. Currently, mutations in more than 60 genes are known to cause non-syndromic RP, and mutations in an additional 73 genes cause syndromic forms of RP, such as Usher syndrome and Bardet-Biedl syndrome. For recessive forms of RP, significant progress has been made in developing gene augmentation therapy, in which supplementing retinal cells with a normal copy of the disease gene can have a therapeutic effect. However, no effective therapy has been developed for dominant RP, which accounts for ˜40% of all RP cases. Most dominant RP genes produce a mutant protein that damages cells, rendering gene augmentation therapy ineffective. A permanent cure for a dominant RP, or any dominant form of inherited retinal degenerations (IRDs), therefore requires correcting or suppressing the production of the mutant protein.

### SUMMARY

CRISPR/Cas9 (Clustered Regularly Interspaced Short Palindromic Repeat-associated protein 9) genome editing technology has been shown to be an efficient tool for editing the genetic code in mammalian cells and many organisms. Described herein are methods for the specific abolishment of the mutant copy of dominant RP genes via CRISPR/Cas9 system to eliminate the production of pathogenic proteins, and thus prevent photoreceptor cell death in dominant RP patients. Thus described herein is the use of CRISPR/Cas9 technology for treating RP1 mutation-associated autosomal dominant Retinitis Pigmentosa (adRP).

Thus, provided herein are nucleic acids comprising sequences encoding a Cas9 protein, and a first guide RNA (gRNA), and a second gRNA, wherein the first and/or second RNAs are targeted to sequences comprising single nucleotide polymorphisms (SNPs) in a mutated allele of a RP1 gene of a subject, wherein the mutated allele is associated with autosomal dominant retinitis pigmentosa (adRP). In some embodiments, the first gRNA comprises a target sequence in intron 1 or 3, and/or wherein the second gRNA comprises a target sequence in intron 3 or exon 4, preferably wherein one or both gRNAs comprise a sequence shown in Table 6. In some embodiments, the first or second gRNAs are targeted to bi-allelic sequences, e.g., in intron 3, while the other gRNA is targeted to a sequence comprising a SNP.

Also provided herein are nucleic acids comprising sequences encoding a Cas9 protein, and optionally at least one gRNA targeted to a sequence comprising a single nucleotide polymorphism (SNP) in intron 1 or 3 or exon 4, and optionally comprising a sequence encoding a second gRNA, optionally wherein the first and second gRNAs targeted to sequences in intron 1 or 3 and/or in intron 3 or exon 4, wherein the second gRNA is targeted to a bi-allelic sequence or sequence comprising a SNP of an RP1 gene of a human subject. In some embodiments, the gRNA comprises a target sequence shown in Table 6.

In some embodiments, the nucleic acids encodes S. aureus Cas9 or S. pyogenes Cas9.

In some embodiments, the Cas9 comprises a nuclear localization signal, optionally a C-terminal nuclear localization signal and/or an N-terminal nuclear localization signal; and/or wherein the sequences encoding Cas9 comprises a polyadenylation signal.

In some embodiments, the gRNA is a unimolecular S. aureus gRNA comprising SEQ ID NO: 1 or SEQ ID NO: 2 (which correspond to SEQ ID NO: 7 or SEQ ID NO: 8 of WO 2018/026976 respectively), or the corresponding two-part modular S. aureus gRNA, wherein the crRNA component comprises SEQ ID NO: 3 or SEQ ID NO: 4 (the bold section of SEQ ID NO: 1 or SEQ ID NO: 2) and the tracrRNA component comprises SEQ ID NO: 5 or SEQ ID NO: 6 (the underlined section of SEQ ID NO: 1 or SEQ ID NO: 2).

SEQ ID NO: 1: S. aureus gRNA component
[N]₁₆₋₂₄
GTTTTAGTACTCTGGAAACAGAATCTACTAAAACAAGGCAAAATGCC
GTGTTTATCTCGTCAACTTGTTGGCGAGATTTTTT
SEQ ID NO: 2: S. aureus gRNA component
[N]₁₆₋₂₄
GTTATAGTACTCTGGAAACAGAATCTACTATAACAAGGCAAAATGCC
GTGTTTATCTCGTCAACTTGTTGGCGAGATTTTTT
SEQ ID NO: 3: crRNA component
GTTTTAGTACTCTG
SED ID NO: 4: crRNA component
GTTATAGTACTCTG
SEQ ID NO: 5: tracrRNA component
CAGAATCTACTAAAACAAGGCAAAATGCCGTGTTTATCTCGTCAACTTG
TTGGCGAGATTTTTT
SEQ ID NO: 6: tracrRNA component
CAGAATCTACTATAACAAGGCAAAATGCCGTGTTTATCTCGTCAACTTG
TTGGCGAGATTTTTT

In some embodiments, the gRNA is an S. pyogenes gRNA comprising any one of the sequences set forth in SEQ ID NOs: 7-16 (which correspond to SEQ ID NOs: 2404, 2407, 2408, and 1-7 of WO 2014/144592)

SEQ ID NO: 7: S. pyogenes gRNA
(X₁₇₋₂₀)GUUUUAGAGCUA;
SEQ ID NO: 8: S. pyogenes gRNA
(X₁₇₋₂₀)GUUUUAGAGCUAUGCUGUUUUG
SEQ ID NO: 9: S. pyogenes gRNA
(X₁₇₋₂₀)GUUUUAGAGCUAUGCU
SEQ ID NO: 10: S. pyogenes gRNA
(X₁₇₋₂₀)GUUUUAGAGCUAGAAAUAGCAAGUUAAAAUAAGGCUAGUCCG
SEQ ID NO: 11: S. pyogenes gRNA
(X₁₇₋₂₀)
GUUUUAGAGCUAUGCUGAAAAGCAUAGCAAGUUAAAAUAAGGCUAG
UCCGUUAUC
SEQ ID NO: 12: S. pyogenes gRNA
(X₁₇₋₂₀)
GUUUUAGAGCUAUGCUGUUUUGGAAACAAAACAGCAUAGCAAGUUAA
AAUAAGGCUAGUCCGUUAUC
SEQ ID NO: 13: S. pyogenes gRNA
(X₁₇₋₂₀)
GUUUUAGAGCUAGAAAUAGCAAGUUAAAAUAAGGCUAGUCCGUUAU
CAACUUGAAAAAGUGGCACCGAGUCGGUGC
SEQ ID NO: 14: S. pyogenes gRNA
(X₁₇₋₂₀)
GUUUAAGAGCUAGAAAUAGCAAGUUUAAAUAAGGCUAGUCCGUUAUC
AACUUGAAAAAGUGGCACCGAGUCGGUGC
SEQ ID NO: 15: S. pyogenes gRNA
(X₁₇₋₂₀)
GUUUUAGAGCUAUGCUGGAAACAGCAUAGCAAGUUUAAAUAAGGCUAG
UCCGUUAUCAACUUGAAAAAGUGGCACCGAGUCGGUGC
SEQ ID NO: 16: S. pyogenes gRNA
(X₁₇₋₂₀)
GUUUAAGAGCUAUGCUGGAAACAGCAUAGCAAGUUUAAAUAAGGCUAG
UCCGUUAUCAACUUGAAAAAGUGGCACCGAGUCGGUGC

wherein X₁₇₋₂₀ is a complementarity region that is complementary to 17-20 consecutive nucleotides of the complementary strand of a selected target sequence, preferably a target sequence immediately 5′ a protospacer adjacent motif (PAM).

In some embodiments, the nucleic acids comprise a vector, e.g., a viral delivery vector. In some embodiments, the viral delivery vector comprises a promoter for Cas9, e.g., a CMV, EFS, or hGRKl promoter. In some embodiments, the viral delivery vector comprises a promoter for the gRNA, e.g., a U6 promoter.

In some embodiments, the viral delivery vector comprises an adeno-associated virus (AAV) vector.

In some embodiments, the nucleic acids comprise (i) a first guide RNA (gRNA) comprising a sequence targeting a domain in intron 1 or 3 of the human RP1 gene, and a second gRNA comprising a sequence targeting a domain in intron 3 or exon 4 of the human RP1 gene, or a single guide RNA comprising a sequence selected from the group listed in Table 6; (ii) a first and a second inverted terminal repeat sequence (ITR); (iii) a promoter, e.g., a U6 promoter, for driving expression of the first and second gRNAs; and (iv) a promoter for driving expression of the Cas9, preferably selected from the group consisting of a CMV, an EFS, or an hGRKl promoter. In some embodiments, the promoter driving expression of a gRNA is an H1 or 7SK promoter.

Also provided are the nucleic acids described herein for use in therapy, as well as the nucleic acids described herein for use in preparation of a medicament, or for use in a method of treating a subject who has a condition associated with a mutation in RP1.

In some embodiments, the condition is autosomal dominant retinitis pigmentosa (adRP).

In some embodiments, the AAV vector is delivered to a retina of a subject by injection, such as by subretinal injection.

Also provided herein are methods for altering the genome of a cell. The methods comprise using CRISPR editing to form a first double strand break within intron 1 or 3 of the human RP1 gene and a second double strand within intron 3 or 4 of the human RP1 gene. In some embodiments, the cell is a cell of the eye of a mammal, e.g., human.

In some embodiments, the first and second double strand breaks are generated using a pair of gRNAs comprising sequences selected from Table 6.

In some embodiments, the first and second double strand breaks are repaired by non-homologous end joining (NHEJ) in a manner that results in removal of all or part of exon 2, exon 3, and exon 4 of a mutant allele of an RP1 gene on chromosome 8, sufficient to disrupt expression of the RP1 protein from that allele.

In some embodiments, the first and second double strand breaks are repaired by non-homologous end joining (NHEJ) in a manner that results in removal of (a) all of exon 2 and exon 3; (b) part of exon 4; or (c) all of exon 2 and exon 3 and part of exon 4; of a mutant allele of an RP1 gene on chromosome 8, sufficient to disrupt expression of the RP1 protein from that allele. In some embodiments, the first and second double strand breaks are repaired by non-homologous end joining (NHEJ) in a manner that results in removal of all or part of exon 2 and exon 3, or exon 4 of a mutant allele of an RP1 gene on chromosome 8, sufficient to disrupt expression of the RP1 protein from that allele.

In some embodiments, the first and/or second gRNA is configured to form a complex with a Cas9 molecule.

In some embodiments, the cell is from a subject suffering from autosomal dominant retinitis pigmentosa (adRP).

In some embodiments, the cell is a retinal cell or a photoreceptor cell.

In some embodiments, the photoreceptor cell is a cone photoreceptor cell or a cone cell, a rod photoreceptor cell or a rod cell or a macular cone photoreceptor cell.

Further, provided herein are methods for altering a cell. The methods include contacting the cell with a recombinant viral particle comprising a nucleotide sequence encoding a first gRNA comprising a sequence targeting a domain in intron 1 or 3 of the human RP1 gene, preferably as shown in Table 6; a nucleotide sequence encoding a second gRNA molecule comprising a sequence targeting a domain in intron 3 or exon 4 of the human RP1 gene, preferably as shown in Table 6; and a nucleotide sequence encoding a Cas9 molecule; wherein said viral particle is capable of delivery to a non-dividing cell, and wherein said contacting results in removal of all or part of exon 2, exon 3, and exon 4 of a mutant allele of an RP1 gene on chromosome 8, sufficient to disrupt expression of the RP1 protein from that allele.

In some embodiments, the methods include contacting the cell with a recombinant viral particle comprising a nucleotide sequence encoding a first gRNA comprising a sequence targeting a domain in intron 1 or 3 of the human RP1 gene, preferably as shown in Table 6; a nucleotide sequence encoding a second gRNA molecule and a second gRNA comprising a sequence targeting a domain in intron 3 or exon 4 of the human RP1 gene, preferably as shown in Table 6; and a nucleotide sequence encoding a Cas9 molecule; wherein said viral particle is capable of delivery to a non-dividing cell, and wherein said contacting results in removal of all or part of exon 2 and exon 3, or exon 4 of a mutant allele of an RP1 gene on chromosome 8, sufficient to disrupt expression of the RP1 protein from that allele.

In some embodiments, the viral particle is an adeno-associated virus (AAV) viral particle.

In some embodiments of the methods and nucleic acids described herein, the first gRNA and the second gRNA are shown in Table 6.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows common SNP-based CRISPR/Cas9 targeting strategy for mutant RP1 alleles. One of the RP1 haplotype has 3 common SNPs in intron 1, 3 SNPs in intron 3, and 4 SNPs in the last exon. Simultaneously targeting of one SNP in intron 1 or 3, and the other SNPs in intron 3 or exon 4, will delete the RP1 genomic regions (top), which will result in knockout of the RP1 allele. An example of combined target of SNP rs436527 in intron 1 and rs444772 in exon 4 is shown at the bottom.

FIGS. 2A-2B show editing efficiencies of RP1 gRNAs in cultured human cells. FIG. 2A shows screening of individual sgRNAs for Haplotype 1 variants. FIG. 2B shows screening of individual sgRNAs for Haplotype 2 and 3 variants. Plasmids encoding individual sgRNAs were co-transfected with Cas9 expressing plasmids (SpCas9-WT and SpCas9-VRQR), into human cell lines carrying hetero- or homozygous RP1 haplotypes. Target spanning polymerase chain reaction (PCR) was performed, and amplicons were subjected to next-generation sequencing (NGS). Editing efficiency was calculated as the percentage of edited reads/total reads per allele.

FIG. 3 shows quantification of genomic deletion in HAP-1 cells treated with pairs of sgRNAs targeting RP1 haplotype 1. Amplicons within the predicted deletion region were quantified by qPCR, relative to unedited samples. Deletion efficiency was calculated from the reduction in amplicon quantity.

FIG. 4 shows quantification of RP1 expression by qRT-PCR in Weri-Rb1 cells treated with pairs of sgRNAs targeting RP1 haplotype 1. Specific pairs of sgRNAs were able to decrease endogenous RP1 expression in heterozygous (H1/H2) Weri-Rb1 cells.

FIGS. 5A-5B show generation of RP1 expression cell lines. FIG. 5A shows screening of RP1 expression in near-haploid (HAP-1) clonal cell-lines engineered with an EF1a promoter insertion. FIG 5B shows RP1 expression level in engineered HAP-1 stable cell-lines relative to Weri-Rb1 cells. RP1 expression was quantified by qRT-PCR. Engineered HAP1-EF1a-RP1 Clone 5-1 has the highest expression of RP1 transcripts, with a four-fold increase over endogenous RP1 expression in Weri-Rb1 cells.

### DETAILED DESCRIPTION

The effects of dominant mutations can arise via gain-of-function, dominant-negative, or haploinsufficiency mechanisms. The primary challenge in developing a gene therapy for autosomal dominant RP (adRP) is that, in addition to delivery of a healthy gene, the mutant gene may also need to be suppressed (Lewin, A.S., et al., (1998). Nat.Med. 4(8): p. 967-971; O'Reilly, M., et al., (2008). Vision Research, 48(3): p. 386-91; and O'Reilly, M., et al., (2007) American Journal of Human Genetics, 81(1): p. 127-35). The second challenge is that, for many IRD genes, the therapeutic dosage falls within a narrow window such that overexpression of the normal gene can lead to retinal degeneration (O'Reilly, M., et al., (2008) Vision Research. 48(3): p. 386-91; Wen, X.H., et al., (2009) Biophys J, 96(3): p. 939-50; and Liu, Q., et al., (2012) PLoS One, 7(8): p. e43251). For example, in a transgenic Rp1 mouse line, a 50% excess of Rp1 over the physiological normal levels causes an RP-like retinal degeneration (Liu, Q., et al., (2012) PLoS One,. 7(8): p. e43251). Therefore, an optimal therapeutic approach for dominant RP would require not only removal of the mutant protein, but also maintenance of endogenous levels of wild-type protein expression. CRISPR/Cas9 based genome editing tools provide a reliable and practical means to conceivably edit the disease-causing mutations in mammalian cells by introducing double strand breaks (DSBs) near the mutation, which are then repaired through the non-homologous end joining (NHEJ) or homology-directed repair (HDR) pathway, leading to the loss or correction of the mutant allele (Yin, H., et al., (2014) Nat Biotechnol, 2014. 32(6): p. 551-3; Ran, F.A., et al., (2015) Nature, 2015. 520(7546): p. 186-91; Swiech, L., et al., (2015) Nat Biotechnol, 2015. 33(1): p. 102-6; Tabebordbar, M., et al., (2016) Science, 2016. 351(6271): p. 407-11; Yang, Y., et al., (2016). Nature Biotechnology, 2016. 34(3): p. 334-8). By directly altering the genomic DNA, the CRISPR/Cas9 system is capable of maintaining the edited gene under its endogenous expression stoichiometry, thereby avoiding ectopic expression and abnormal gene production that may occur with conventional gene augmentation therapies. Recent studies of the RHO form of adRP demonstrated that the Cas9-sgRNA system is capable of distinguishing the single base pair difference between the wild-type and mutant P23H allele of the rhodopsin gene, and thus specifically disrupting the expression of mutant allele in mouse retina. This mutation-based gene knockout approach, however, requires developing an individual Cas9-sgRNA targeting system for each of the pathogenic mutations in a given gene, which limits its application for treating a larger number of patients.

Dominant mutations identified to date in RP1 are either nonsense or frame-shift mutations that cluster at the N-terminal half of the last exon, exon 4 (Gandra, M., et al., (2008). Mol.Vis., 14: p. 1105-1113; Chen, L.J., et al., (2009). Invest Ophthalmol. Vis.Sci. 51(4): p. 2236-2242; Liu, Q., et al, (2009) Investigative ophthalmology & visual science 50(4): p. 1566-74; Liu, Q., et al., (2012). PLoS One, 7(8): p. e43251). The mutant RP1 alleles are predicted to encode truncated proteins that play a dominant negative role in photoreceptors (Liu, Q., et al., (2012). PLoS One, 7(8): p. e43251; Liu, Q., A. et al (2009), Invest Ophthalmol Vis Sci, 50(4): p. 1566-74; and Liu, Q., et al. (2004) J Neurosci, 24(29): p. 6427-36). The most common mutation in RP1, R677X, is present in approximately 3% of patients with adRP in the United States, making it the third most common adRP mutation (Jacobson, S.G., et al., (2000) Investigative ophthalmology & visual science, 41(7): p. 1898-908; Berson, E.L., et al., (2001) Investigative ophthalmology & visual science, 42(10): p. 2217-24; Schwartz, S.B., et al., (2003) Investigative Ophthalmology & Visual Science, 44(8): p. 3593-7; and Gamundi, M.J., et al., (2006) BMC.Med.Genet., 7: p. 35). All dominant RP1 mutations are located in the last exon, and the resulting transcripts from such alleles escape nonsense-mediated decay to produce truncated proteins. Thus, disruption of the mutation by Cas9 cleavage and NHEJ repair will only create a new frameshift or truncating indel in the last exon, resulting in persistence of toxic gene products.

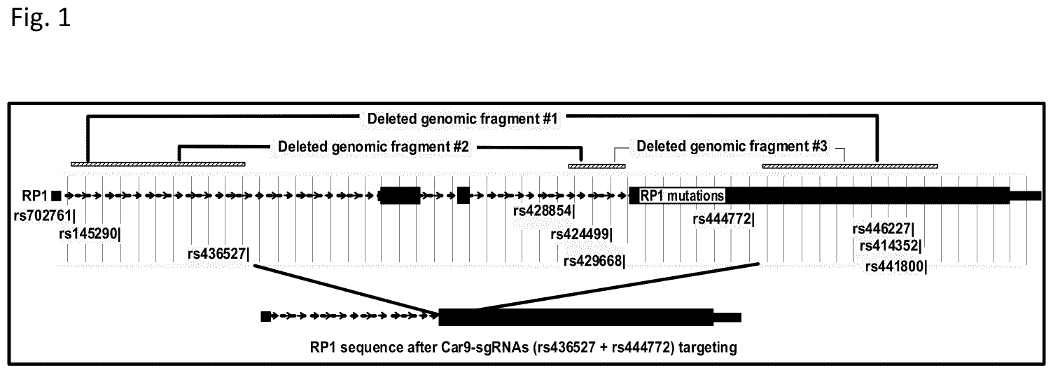

The present methods employ a paired-sgRNA targeting approach using common SNPs as surrogate targeting sites to disrupt the expression of mutant RP1 alleles (FIG. 1). This strategy could be applied to any autosomal dominant mutation in RP1, and would avoid the need for additional gene augmentation therapies, since the wild-type allele remains under its endogenous expression stoichiometry, and 50% of RP1 expression is sufficient for normal retinal integrity and function. The combined frequency of the three most common RP1 haplotypes is 93%, and the total homozygous frequency of the three common haplotypes is 35%. This indicates that approximately two thirds of the general population is heterozygous for RP1 haplotypes, which can be specifically targeted by the SNP-based CRISRP/Cas9 gene editing technology.

Thus, provided herein are methods and compositions for use in treating subjects who have dominant mutations in RP1.

### RNA-Guided Nucleases (RGNs) and Guide RNAs

The present methods include the use of paired RNA-guided nucleases (RGNs) targeted to SNPs in a mutant allele of RP1, to excise portions of and disrupt the expression of the mutant RP1 alleles. The methods can include the use of RGNs including Cas9, Cpf1, and orthologs thereof.

The Cas9 nuclease from S. pyogenes can be guided via simple base pair complementarity between 17-20 nucleotides of an engineered guide RNA (gRNA), e.g., a single guide RNA or crispr RNA/trans-activated crispr RNA (crRNA/tracrRNA) pair, and the complementary strand of a target genomic DNA sequence of interest that lies next to a protospacer adjacent motif (PAM), e.g., a PAM matching the sequence NGG or NAG (Shen et al., (2013) Cell Res; Dicarlo et al., (2013) Nucleic Acids Res; Jiang et al., (2013) Nat Biotechnol 31, 233-239; Jinek et al., (2013) Elife 2, e00471; Hwang et al., (2013) Nat Biotechnol 31, 227-229; Cong et al., (2013) Science 339, 819-823; Mali et al., (2013) Science 339, 823-826; Cho et al., (2013) Nat Biotechnol 31, 230-232; Jinek et al., (2012) Science 337, 816-821). See, e.g., the sequences set forth in SEQ ID NOs: 7-16. The engineered CRISPR from Prevotella and Francisella 1 (Cpf1, also known as Cas12a) nuclease can also be used, e.g., as described in Zetsche et al., (2015) Cell 163, 759-771; Schunder et al., (2013) Int J Med Microbiol 303, 51-60; Makarova et al., (2015) Nat Rev Microbiol 13, 722-736; Fagerlund et al., (2015) Genome Biol 16, 251. Unlike SpCas9, Cpf1/Cas12a requires only a single 42-nt crRNA, which has 23 nt at its 3′ end that are complementary to the protospacer of the target DNA sequence (Zetsche et al., (2015) Cell 163, 759-771). Furthermore, whereas SpCas9 recognizes an NGG PAM sequence that is 3′ of the protospacer, AsCpf1 and LbCp1 recognize TTTN PAMs that are found 5′ of the protospacer (Id.).

In some embodiments, the present system utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., (2016) Nat Biotechnol.Aug;34(8):869-74; Tsai and Joung, (2016) Nat Rev Genet. May;17(5):300-12; Kleinstiver et al., (2016) Nature. Jan 28;529(7587):490-5; Shmakov et al., (2015) Mol Cell. Nov 5;60(3):(2015) 385-97; Kleinstiver et al., Nat Biotechnol. Dec;33(12):1293-1298; Dahlman et al., (2015) Nat Biotechnol. Nov;33(11):1159-61; Kleinstiver et al., (2015) Nature. Jul 23;523(7561):481-5; Wyvekens et al., (2015) Hum Gene Ther. Jul;26(7):425-31; Hwang et al., (2015) Methods Mol Biol. 1311:317-34; Osborn et al., (2015) Hum Gene Ther. Feb;26(2):114-26; Konermann et al., (2015) Nature. Jan 29;517(7536):583-8; Fu et al., (2014) Methods Enzymol. 546:21-45; and Tsai et al., (2014) Nat Biotechnol. Jun;32(6):569-76, inter alia. Exemplary guide RNAs used with S. aureus include those set forth in SEQ ID NOs: 1-6.

Cas9 and analogs are shown in Table 1, and engineered protospacer-adjacent motif (PAM) or high-fidelity variants are shown in Table 2.

TABLE 1
List of Exemplary Cas9 or Cas12a Orthologs

| | UniProt or |
|---|---|
| | GenBank |
| | Accession |
| Ortholog | Number |
| S. pyogenes Cas9 (SpCas9) | Q99ZW2.1 |
| S. aureus Cas9 (SaCas9) | J7RUA5.1 |
| S. thermophilus Cas9 (St1Cas9) | G3ECR1.2 |
| S. pasteurianus Cas9 (SpaCas9) | BAK30384.1 |
| C. jejuni Cas9 (CjCas9) | Q0P897.1 |
| F. novicida Cas9 (FnCas9) | A0Q5Y3.1 |
| P. lavamentivorans Cas9 (PlCas9) | A7HP89.1 |
| C. lari Cas9 (ClCas9) | G1UFN3.1 |
| Pasteurella multocida Cas9 | Q9CLT2.1 |
| F. novicida Cpf1 (FnCpf1) | A0Q7Q2.1 |
| M. bovoculi Cpf1 (MbCpf1) | WP_052585281.1 |
| A. sp. BV3L6 Cpf1 (AsCpf1) | U2UMQ6.1 |
| L. bacterium N2006 (LbCpf1) | A0A182DWE3.1 |

TABLE 2
List of Exemplary High Fidelity and/or PAM-relaxed RGN Orthologs

| Published | | |
|---|---|---|
| HF/PAM-RGN | PMID/US | |
| variants | PGPUB | Mutations* |
| S. pyogenes Cas9 | 26628643 | K810A/K1003A/R1060A (1.0); |
| (SpCas9) | | K848A/K1003A/R1060A (1.1) |
| eSpCas9 | | |
| S. pyogenes Cas9 | 29431739 | M495V/Y515N/K526E/R661Q; |
| (SpCas9) | | (M495V/Y515N/K526E/R661S; |
| evoCas9 | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi | | |
| Cas9 | | |
| S. pyogenes Cas9 | 28931002 | N692A, M694A, Q695A, H698A |
| (SpCas9) | | |
| HypaCas9 | | |
| S. pyogenes Cas9 | 30082838 | F539S, M763I, K890N |
| (SpCas9) Sniper- | | |
| Cas9 | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) | | E1219F, A1322R, T1337R |
| SpCas9-NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | US 20190010481 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R |
| enAsCas 12a-HF | US 20190010481 | One or more of: E174R, S542R, K548R, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R, with the addition of |
| | | one or more of: N282A, T315A, N515A and K949A |
| | US 20190010481 | One or more of T152R, T152K, D156R, D156K, |
| | | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, |
| | | D156R/G532R/K538V/Y542R, |
| enLbCas 12a(HF) | | T152R/G532R/K538R, T152R/D156R, |
| | | D156R/G532R, T152R/G532R, |
| | | D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | US 20190010481 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |

*predicted based on UniRule annotation on the UniProt database.

In some embodiments an RGN sequence is modified to include a nuclear localization sequences (NLSs), e.g., at the C- and/or N-terminus of the RGN protein, and a mini-polyadenylation signal (or Poly-A sequence). Exemplary NLSs include SV40 large T antigen NLS (PKKKRRV (SEQ ID NO: 17)); PKKKRKV (SEQ ID NO: 18); KRTADGSEFESPKKKRKV (SEQ ID NO: 19); and nucleoplasmin NLS (KRPAATKKAGQAKKKK (SEQ ID NO: 20)). Other NLSs are known in the art; see, e.g., Cokol et al., (2000) EMBO Rep. Nov 15; 1(5):411-415; Freitas and Cunha, (2009) Curr Genomics. Dec; 10(8): 550-557; and Leung et al (2003) Journal of Biol Chem. 278(43):41947-41953. An exemplary polyadenylation signal is

(SEQ ID NO: 21))
TAGCAATAAAGGATCGTTTATTTTCATTGGAAGCGTGTGTTGGTTTTTTG
ATCAGGCGCG.

Guide RNAs appropriate for the RGN should be used. In some embodiments, the gRNAs used in the present disclosure can be unimolecular or modular, as known in the art.

### Vectors

Sequences encoding the RGN and guide RNA can be delivered to the retina, e.g., using a viral vector. Described herein are targeted expression vectors for in vivo transfection and expression of a polynucleotide that encodes a RGN and guide RNAs as described herein, in the retina, e.g., in photoreceptors, e.g., primarily or only in photoreceptors. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, alphavirus, vaccinia virus, or recombinant bacterial or eukaryotic plasmids; preferred viral vectors are adeno-associated virus type 2 (AAV2), AAV5 or AAV8. Viral vectors transfect cells directly; plasmid DNA can be delivered naked or with the help of, for example, cationic liposomes (lipofectamine) or derivatized (e.g., antibody conjugated), cationic dendrimers, inorganic vectors (e.g., iron oxide magnetofection), lipidoids, cell-penetrating peptides, cyclodextrin polymer (CDP), polylysine conjugates, gramacidin S, artificial viral envelopes or other such intracellular carriers, as well as direct injection of the gene construct or CaPO4 precipitation carried out in vivo.

An exemplary approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Viral vectors can be used as a recombinant gene delivery system for the transfer of exogenous genes in vivo, particularly into humans. These vectors provide efficient delivery of genes into cells, and in some cases the transferred nucleic acids are stably integrated into the chromosomal DNA of the host. Protocols for producing recombinant viruses and for infecting cells in vitro or in vivo with such viruses can be found in Ausubel, et al., eds., Gene Therapy Protocols Volume 1: Production and In Vivo Applications of Gene Transfer Vectors, Humana Press, (2008), pp. 1-32 and other standard laboratory manuals.

A preferred viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol.158:97-129 (1992); see also Domenger and Grimm, Human Molecular Genetics, 28(R1):R3-R14 (October 2019)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 Aug; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989)). AAV vectors, particularly AAV2, have been extensively used for gene augmentation or replacement and have shown therapeutic efficacy in a range of animal models as well as in the clinic; see, e.g., Mingozzi and High, (2011) Nature Reviews Genetics 12, 341-355; Deyle and Russell, (2009) Curr Opin Mol Ther. Aug; 11(4): 442-447; Asokan et al., (2012) Mol Ther. April; 20(4): 699-708. AAV vectors containing as little as 300 base pairs of AAV can be packaged and can produce recombinant protein expression. Space for exogenous DNA is limited to about 4.5 kb. For example, an AAV1, 2, 4, 5, or 8 vector can be used to introduce DNA into the retina, e.g., into photoreceptors, inner retinal cells, or RPE cells (such as those described in Maguire et al. (2008).. N Engl J Med 358:2240-2248; Maguire et al. (2009). Lancet 374:1597-1605; Bainbridge et al. (2008). N Engl J Med 358:2231-2239; Hauswirth et al. (2008). Hum Gene Ther 19:979-990; Cideciyan et al. (2008). Proc Natl Acad Sci USA 105:15112-15117; Cideciyan et al. (2009). N Engl J Med 361: 725-727; Simonelli et al. (2010). Mol Ther 18:643-650; Acland, et al. (2005). Mol Ther 12:1072-1082; Le Meur et al. (2007). Gene Ther 14:292-303; Stieger et al. (2008). Mol Ther 16:916-923; and Vandenberghe et al. (2011). Sci Transl Med 3: 88ra54). In some embodiments, the AAV vector can include (or include a sequence encoding) an AAV capsid polypeptide described in WO 2015054653; for example, a virus particle comprising an AAV capsid polypeptide having an amino acid sequence selected from the group consisting of SEQ ID NOs: 1, 3, 5, 7, 9, 11, 13, 15, and 17 of WO 2015054653, and a RGN and guide RNA-encoding sequences as described herein. In some embodiments, the AAV capsid polypeptide is as shown in Table 1 of WO 2015054653, reproduced here:

| | Polypeptide | Nucleic Acid |
|---|---|---|
| Node | (SEQ ID NO) | (SEQ ID NO) |
| Anc80 | 1 | 2 |
| Anc81 | 3 | 4 |
| Anc82 | 5 | 6 |
| Anc83 | 7 | 8 |
| Anc84 | 9 | 10 |
| Anc94 | 11 | 12 |
| Anc113 | 13 | 14 |
| Anc126 | 15 | 16 |
| Anc127 | 17 | 18 |

In some embodiments, the AAV capsid polypeptide is an Anc80 polypeptide, e.g., an exemplary polypeptide shown in SEQ ID NO: 19 (Anc80L27); SEQ ID NO: 20 (Anc80L59); SEQ ID NO: 21 (Anc80L60); SEQ ID NO: 22 (Anc80L62); SEQ ID NO: 23 (Anc80L65); SEQ ID NO: 24 (Anc80L33); SEQ ID NO: 25 (Anc80L36); and SEQ ID NO: 26 (Anc80L44) of WO 2015054653.

A variety of nucleic acids have been introduced into different cell types using AAV vectors (see for example the references cited above and those cited in Asokan et al., (2012) Molecular Therapy; 20 4, 699-708; and Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790.

In some embodiments, a self-complementary AAV is used, which contains an inverted repeat genome that folds to make double-stranded DNA.

Retroviruses can also be used. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Katz et al., (2013) Human Gene Therapy 24:914). A replication defective retrovirus can be packaged into virions, which can be used to infect a target cell through the use of a helper virus by standard techniques. Examples of suitable retroviruses include pLJ, pZIP, pWE and pEM, which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest, but is inactivated in terms of its ability to replicate in a normal lytic viral life cycle. See, for example, Berkner et al., (1988) BioTechniques 6:616; Rosenfeld et al., (1991) Science 252:431-434; and Rosenfeld et al., (1992) Cell 68:143-155. Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, (1986) J. Virol. 57:267.

In some embodiments, sequences encoding RGN and guide RNAs is entrapped in liposomes bearing positive charges on their surface (e.g., lipofectins), which can be tagged with antibodies against cell surface antigens of the target tissue (Mizuno et al., (1992) No Shinkei Geka 20:547-551; PCT publication WO91/06309; Japanese patent application 1047381; and European patent publication EP-A-43075).

The vectors can also include promoters, enhancers (e.g., CMV enhancer), other cis-regulatory elements, and/or capsid serotype variants. With regard to promoters, vectors can include promoters that drive expression in many cell types (e.g., cytomegalovirus (CMV), chicken β-actin (CBA), cytomegalovirus (CMV) enhancer fused to the chicken beta-actin promoter (CAG), CBh, elongation factor alpha 1 (EFalpha1), EF-1 Alpha Short (EFS) or CASI) or specifically in photoreceptor cells (e.g., RHO, beta phosphodiesterase (PDE), retinitis pigmentosa (RP1), rhodopsin kinase (hGRK1) and cone arrestin (CAR)) (see, e.g., Gray et al., (2011) Hum Gene Ther. Sep;22(9):1143-53; Alexopoulou et al., (2008) BMC Cell Biol.; 9:2; Esumi et al., (2004) Journal Biological Chemistry; 279:19064-73; Guziewicz et al., (2013) PLoS One.; 8: e75666; Allocca et al., (2007) J Virol; 81:11372-80; see also Domenger and Grimm, (2019) Human Molecular Genetics, 28(R1):R3-R14 (October)). Other cis-regulatory elements can include enhancer elements derived from the interphotoreceptor retinoid-binding protein gene (IRBP), woodchuck hepatitis virus post-transcriptional regulatory element (WPRE), or minute virus of mice (MVM) intron (see Domenger and Grimm, (2019) Human Molecular Genetics, 28(R1):R3-R14 (October)).

The pharmaceutical preparation of the gene therapy construct can consist essentially of the gene delivery system (viral vector and any associated agents such as helper viruses, proteins, lipids, and so on) in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

Alternatively, the methods can include delivering the RGN and guide RNA together, e.g., as a complex. For example, the RGN and gRNA can be can be overexpressed in a host cell and purified, then complexed with the guide RNA (e.g., in a test tube) to form a ribonucleoprotein (RNP), and delivered to cells. In some embodiments, the RGN can be expressed in and purified from bacteria through the use of bacterial expression plasmids. For example, His-tagged deaminase fusion protein can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there is no persistent expression of the nuclease and guide (in contrast to the sustained expression from a plasmid). The RNPs can be delivered to the cells in vivo or in vitro, e.g., using lipid-mediated transfection or electroporation. See, e.g., Liang et al. (2015) Journal of biotechnology 208:44-53; Zuris, John A., et al. (2015) Nature biotechnology 33.1:73-80; Kim et al. (2014) Genome research 24.6:1012-1019.

### Methods of Treatment

Provided herein are methods of treating subjects (e.g., mammalian subjects, e.g., human or non-human veterinary subjects) who have Retinitis pigmentosa (RP) associated with dominant mutations in the RP1 gene. Suitable subjects can be identified by one of skill in the art and a diagnosis confirmed by genetic testing (e.g., sequencing to identify the presence of a mutation in the subject's RP1 gene). A reference sequence for RP1 can be found in NCBI GenBank at RefSeqGene ID NG_009840.2 (Range 5030-19768).

The methods include administering an effective amount of an RGN and paired guide RNAs that excise portions of and disrupt the expression of the mutant RP1 alleles. In clinical settings, the vectors can be introduced into a subject by any of a number of methods, each of which familiar in the art. Although other methods can be used, in some embodiments, the route of choice for delivery of gene therapy vectors to the retina is via sub-retinal injection. This provides access to the RPE and photoreceptor cells of the retina. Different serotypes of AAV have been shown to transduce these cell populations effectively after sub-retinal injection in animal studies (Vandenberghe et al., (2013) PLoS One. 8:e53463. PMCID: 3559681; Vandenberghe and Auricchio, (2012) Gene Therapy. 19:162-8; Vandenberghe et al., (2011) Science translational medicine. 3:88ra54; Dinculescu et al., (2005) HumGene Ther. 16:649-63; Boye et al., (2013) Mol Ther. 21:509-19; and Alexander and Hauswirth, (2008) Adv Exp Med Biol. 2008;613:121-8). The sub-retinal injection approach is being used in the ongoing clinical trials of gene augmentation therapy for retinal degeneration caused by mutations in the RPE65 and CHM genes (Maguire et al., (2008) New England Journal of Medicine. 358:2240-8; Bainbridge et al., (2008) New England Journal of Medicine. 358:2231-9; Cideciyan et al., (2008) Proceedings National Academy Sciences USA. 105:15112-7; Maguire et al., (2009) Lancet. 374:1597-605; Jacobson et al., (2012) Archives Ophthalmology. 130:9-24; Bennett et al., (2012) Science translational medicine. 4:120ra15; and MacLaren et al., (2014) Lancet. 383:1129-37). Sub-retinal injections can be performed using a standard surgical approach (e.g., as described in Maguire et al., 2008 supra; Bainbridge et al., 2008 supra; Cideciyan et al., 2008 supra; MacLaren et al., 2014 supra). See also WO2019/183641.

Any region of the retina may be targeted, though the fovea (which extends approximately 1 degree out from the center of the eye) may be preferred in certain instances due to its role in central visual acuity and the relatively high concentration of cone photoreceptors there relative to peripheral regions of the retina. Alternatively or additionally, injections may be targeted to parafoveal regions (extending between approximately 2 and 10 degrees off center), which are characterized by the presence of all three types of retinal photoreceptor cells. In addition, injections into the parafoveal region may be made at comparatively acute angles using needle paths that cross the midline of the retina. For instance, injection paths may extend from the nasal aspect of the sclera near the limbus through the vitreal chamber and into the parafoveal retina on the temporal side, from the temporal aspect of the sclera to the parafoveal retina on the nasal side, from a portion of the sclera located superior to the cornea to an inferior parafoveal position, and/or from an inferior portion of the sclera to a superior parafoveal position. The use of relatively small angles of injection relative to the retinal surface may advantageously reduce or limit the potential for spillover of vector from the bleb into the vitreous body and, consequently, reduce the loss of the vector during delivery. In other cases, the macula (inclusive of the fovea) can be targeted, and in other cases, additional retinal regions can be targeted, or can receive spillover doses.

Compositions comprising AAV vectors can be administered to subjects by any suitable means, including without limitation injection, for example, sub-retinal injection. The concentration of AAV vector within the composition is selected to ensure, among other things, that a sufficient AAV dose is administered to the retina of the subject, taking account of dead volume within the injection apparatus and the relatively limited volume that can be safely administered. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the subretinal or cochlear space. In some instances, the volume is selected to form a bleb in the subretinal space, for example 1 microliter, 10 microliters, 50 microliters, 100 microliters, 150 microliters, 200 microliters, 250 microliters, 300 microliters, etc.

Further provided herein are viruses comprising sequences encoding an RGN and guide RNAs as described herein (e.g., one or more viruses comprising sequences one, two, or all three of the RGN and paired gRNAs, e.g., wherein a single virus comprises sequences encodes one, two (e.g., two gRNAs), or all three, as well as compositions comprising one or more of such viruses (e.g., a composition comprising one virus comprising sequences encoding the two gRNAs and a separate virus comprising a sequence encoding the RGN; a composition comprising one virus comprising sequences encoding the two gRNAs and the RGN; or a composition comprising one virus comprising a sequence encoding a first gRNA, a second virus comprising a sequence encoding a second gRNA, and a third virus comprising a sequence encoding the RGN). Also provided are RNPs comprising the RGN and gRNAs, and compositions comprising RNPs comprising the RGN and each of the gRNAs.

### EXAMPLES

The invention is further described in the following examples, which do not limit the scope of the invention described in the claims.

### SNP-Based CRISPR/Cas9 Gene-Editing System for RP1 Mutations

The three most common RP1 haplotypes (H1, H2 and H3, identified from the 1000 Genomes Phase1 variants for protein coding regions) occur with a frequency of 42.5%, 27% and 23.5%, respectively (Table 3). H1 is the reference sequence. Common SNPs on H2 include G at rs702761, G at rs145290, A at rs436527, A at rs428854, C at rs424499, C at rs429668, A at rs444772, A at rs446227, C at rs414352, and G at rs441800.Common SNPs on H3 include A for rs62514616, T for rs2293869, and A for rs61739567. The combined frequency of these three common haplotypes is 93% and the total homozygous frequency of the three common haplotypes is 35%. This indicates that approximately two thirds of the general population is heterozygous for RP1 haplotypes, which can be specifically targeted by the SNP-based CRISPR/Cas9 gene editing technology.

TABLE 3
RP1 haplotypes from 1000 Genome Project

| | Haplotype | Homozygous | | | | | | | | | |
|---|---|---|---|---|---|---|---|---|---|---|---|
| | Freq | Freq | 1 | 2 | 3 | 4 | 5 | 6 | 7 | 8 | 9 |
| Haplotypes | % | % | rs702761 | rs145290 | rs436527 | rs428854 | rs424499 | rs429668 | rs444772 | rs446227 | rs414352 |
| H1 (Ref) | 42.5 | 19.1 | A | A | G | G | T | T | G | G | T |
| H2 | 27 | 8.42 | G | G | A | A | C | C | A | A | C |
| H3 | 23.5 | 7.51 | A | A | G | G | T | T | G | G | T |

| | | Homo- | | | | |
|---|---|---|---|---|---|---|
| | Haplotype | zygous | | | | |
| Haplo- | Freq | Freq | 10 | 11 | 12 | 13 |
| types | % | % | rs441800 | rs62514616 | rs2293869 | rs61739567 |
| H1 (Ref) | 42.5 | 19.1 | A | G | A | G |
| H2 | 27 | 8.42 | G | G | A | G |
| H3 | 23.5 | 7.51 | A | A | T | A |

The allele frequencies for the four common SNPs (rs444772, rs446227, rs414352 and rs441800) on H2 is 0.25-0.27 in European Americans based on the 1000 Genomes Project (Table 4). The two SNPs (rs2293869 and rs61739567) for H3 have an allele frequency of 0.44 in European Americans. To reveal the common SNPs in non-coding regions for H2 and H3, we performed a linkage disequilibrium (LD) analysis using HaploReg v4.1. We found that three SNPs (rs702761, rs145290 and rs436527) in intron 1 and three SNPs (rs428854, rs424499 and rs429668) in intron 3 had extremely tight linkage (D′=0.99 or 1) with the 4 common SNPs on H2 (Table 4). Several SNPs in the regulatory region are also tightly linked to H2. One SNP rs62514616 is tightly linked with the two common SNPs on H 3. These SNPs are all potential surrogates for our CRISPR/Cas9 editing approach that is described below.

TABLE 4
SNPs that are in tight linkage on RP1 haplotype 1 and 2

| | Distance | | | | | | | | | | | |
|---|---|---|---|---|---|---|---|---|---|---|---|---|
| | to | | | | AF | | | | | | | |
| SNPs | R677* | Location | Ref | Alt | (EUR) | rs702761 | rs145290 | rs436527 | rs428854 | rs424499 | rs429668 | rs444772 |
| rs702761 | −9518 | Intron 1 | A | G | 0.27 | 1 | 1 | 1 | 1 | 1 | 1 | 1 |
| rs145290 | −8859 | Intron 1 | A | G | 0.26 | 1 | 1 | 1 | 1 | 1 | 1 | 1 |
| rs436527 | −6954 | Intron 1 | G | A | 0.25 | 1 | 1 | 1 | 1 | 0.99 | 0.99 | 0.99 |
| rs428854 | −2101 | Intron 3 | G | A | 0.25 | 1 | 1 | 1 | 1 | 0.99 | 0.99 | 0.99 |
| rs424499 | −1552 | Intron 3 | T | C | 0.26 | 1 | 1 | 0.99 | 0.99 | 1 | 1 | 1 |
| rs429668 | −1333 | Intron 3 | T | C | 0.26 | 1 | 1 | 0.99 | 0.99 | 1 | 1 | 1 |
| rs444772 | 586 | exon 4 | G | A | 0.26 | 1 | 1 | 0.99 | 0.99 | 1 | 1 | 1 |
| rs446227 | 2979 | exon 4 | G | A | 0.25 | 1 | 1 | 1 | 1 | 1 | 1 | 1 |
| rs414352 | 3042 | exon 4 | T | C | 0.26 | 1 | 1 | 0.99 | 0.99 | 1 | 1 | 1 |
| rs441800 | 3146 | exon 4 | A | G | 0.25 | 1 | 1 | 1 | 1 | 1 | 1 | 1 |

| | Distance | | | | | | | |
|---|---|---|---|---|---|---|---|---|
| | to | | | | AF | | | |
| SNPs | R677* | Location | Ref | Alt | (EUR) | rs446227 | rs414352 | rs441800 |
| rs702761 | −9518 | Intron 1 | A | G | 0.27 | 1 | 1 | 1 |
| rs145290 | −8859 | Intron 1 | A | G | 0.26 | 1 | 1 | 1 |
| rs436527 | −6954 | Intron 1 | G | A | 0.25 | 1 | 0.99 | 1 |
| rs428854 | −2101 | Intron 3 | G | A | 0.25 | 1 | 0.99 | 1 |
| rs424499 | −1552 | Intron 3 | T | C | 0.26 | 1 | 1 | 1 |
| rs429668 | −1333 | Intron 3 | T | C | 0.26 | 1 | 1 | 1 |
| rs444772 | 586 | exon 4 | G | A | 0.26 | 1 | 1 | 1 |
| rs446227 | 2979 | exon 4 | G | A | 0.25 | 1 | 1 | 1 |
| rs414352 | 3042 | exon 4 | T | C | 0.26 | 1 | 1 | 1 |
| rs441800 | 3146 | exon 4 | A | G | 0.25 | 1 | 1 | 1 |

*The distances shown are all relative to the most common R677X mutation in RP1 gene. D’ is shown for each pair of SNPs.

From the IRD Biobank at MEEI, we collected 50 affected individuals from 38 families with dominant mutations in the RP1 gene. The most common mutation R677X was detected in 15 patients from 13 families. To determine the phase of the mutation with the SNPs of a given RP1 haplotype, we first analyzed the diploid genotype of RP1 using the NGS-based Genetic Eye Disease (GEDi) test results available from 20 RP1 patients. As shown in Table 5, 14 out of 20 patients are heterozygous at the RP1 locus, and 6 are homozygous for one of the three common haplotypes. The 19 unique dominant mutations that we found in our 38 RP1 families are all clustered in a small region at the beginning of exon 4 (c.1199, p.Q400-c.2287, p. N763). We inspected the paired-end 2×150 bp NGS reads and identified that the 4 bp deletion mutation (c.2280_2284del) in sample 004-235 is in trans with rs444772 on Hap2, indicating that the mutation is on the same allele with Hap3 alleles. Thus, sgRNAs designed to target the SNPs on the Hap3 will be able to abolish the mutant allele in sample 004-235. However, the short NGS reads in the GEDi test could not determine the phase of other mutations with rs444772 due to the distance.

TABLE 5
RP1 haplotypes in adRP patients

| Sample | | | | | | | | | | |
|---|---|---|---|---|---|---|---|---|---|---|
| ID | RP1 Mutation | Protein | Haplotypes | Diploid | rs444772 | rs2293869 | rs446227 | rs414352 | rs441800 | rs61739567 |
| OGI1188_002303 | c.1199_1200del | p.Q400NA | H2/H3 | het | het | het | het | het | het | het |
| OGI785_001529 | c.1468G > T | p.E490* | H3/H3 | hom | — | hom | — | — | — | hom |
| D00760_000782 | c.1555C > T | p.Q519* | H1/H3 | het | — | het | — | — | — | het |
| OGI712_001422 | c.1621_1621del | p.K541NA | H2/H2 | hom | hom | | hom | hom | hom | |
| D00827_000892 | c.1625C > G | p.S542* | H1/H1 | hom | — | — | — | — | — | — |
| JB431 | c.2029C > T | p.R677* | H2/H3 | het | het | het | het | het | het | het |
| JB44 | c.2029C > T | p.R677* | H2/H3 | het | het | het | het | het | het | het |
| OGI680_001363 | c.2029C > T | p.R677* | H1/H2 | het | het | — | het | het | het | — |
| D00827_000892 | c.2039_2040insA | p.A680NA | H1/H1 | hom | — | — | — | — | — | — |
| D01581_002136 | c.2039_2040insA | p.A680NA | H1/H1 | hom | — | — | — | — | — | — |
| OGI741_001462 | c.2056C > T | p.Q686* | H3/H3 | hom | — | hom | — | — | — | hom |
| D00520_000364 | c.2103_2106del | p.R701NA | H1/H3 | het | — | het | — | — | — | het |
| D227_1 | c.2103_2106del | p.R701NA | H1/H3 | het | — | het | — | — | — | het |
| 001-106 | c.2143C > T | p.Q715* | H2/H3 | het | het | het | het | het | het | het |
| FB000016 | c.2167G > T | p.G723* | H2/H3 | het | het | het | het | het | het | het |
| D01692_002340 | c.2219C > G | p.S740* | H1/H3 | het | — | het | — | — | — | het |
| OGI-327-776 | c.2219C > G | p.S740* | H1/H2 | het | het | — | het | het | het | — |
| OGI1153_002251 | c.2271_2271del | p.F757NA | H2/H3 | het | het | het | het | het | het | het |
| 004-235 | c.2280_2284del | p.N760NA | H2/H3 | het | het | het | het | het | het | het |
| D01627_002212 | c.2296C > T | p.Q766* | H1/H2 | het | het | — | het | het | het | — |

### Design of Cas9-sgRNAs for Targeting SNPs on RP1 Haplotypes

For each of the SNPs, we searched available PAM motifs for wild-type SpCas9 (NGG), an engineered VRQR variant of SpCas9 (NGAN), wild-type Staphylococcus aureus Cas9 (SaCas9-WT, NNGRRT), and an engineered KKH variant of SaCas9 (NNNRRT). We designed 1-7 sgRNAs to target each SNP site within 13 bp proximal to the PAM or falling in the PAM (bolded) on the reference haplotype (Table 6). For guides targeted to H2 and H3, we switched the reference nucleotide (shown in bold) to the SNP nucleotide. Four gRNAs targeting the wild type genome in intron 3 were also designed.

TABLE 6
Cas9-sgRNAs for targeting SNPs on human RP1 haplotypes

| | | | | | | | | Targeted |
|---|---|---|---|---|---|---|---|---|
| | | | | sgRNA | | | | haplo- |
| Site | SNPs | Seq | Cas9 | ID | Protospacers | # | PAM | types |
| 1 | rs702761 | A/G | Sp-WT | 1A1 and 1G1 | ATGCTGATATTGAAR | 22 | TGG | H1 or |
| | | | | | TAGAA | | | H3/H2 |
| | | | Sp-WT | 1A2 and 1G2 | TGCTGATATTG | 23 | GGAA | H1 or |
| | | | | | AARTAGAAT | | | H3/H2 |
| | | | Sp-VRQR | 1A3 and 1G3 | CAATATGCTGAT | 24 | AGAA | H1 or |
| | | | | | ATTGAART | | | H3/H2 |
| | | | Sa-WT | 1A4 and 1G4 | TCACAATATGCT | 25 | TAGAAT | H1 or |
| | | | | | GATATTGAAR | | | H3/H2 |
| | | | Sa-KKH | 1A5 and 1G5 | TGATATTGAARTAG | 26 | TTCAGT | H1 or |
| | | | | | AATGGAAC | | | H3/H2 |
| | | | Sa-KKH | 1A6 and 1G6 | TGATACTGAAGTT | 27 | TTCAAT | H1 or |
| | | | | | CCATTCTAY | | | H3/H2 |
| 2 | rs145290 | A/G | Sp-VRQR | 2G1 | CATTGTCTAACTTTA | 28 | TGAA | H2 |
| | | | | | GGCAA | | | |
| | | | Sp-VRQR | 2A2 and 2G2 | TGTCTAACTTTAGGC | 29 | AGAG | H1 or |
| | | | | | AATRA | | | H3/H2 |
| | | | Sp-VRQR | 2A3 and 2G3 | AGGCAATRAAGAGTC | 30 | AGAN | H1 or |
| | | | | | TCTGC | | | H3/H2 |
| | | | Sa-WT | 2A4 and 2G4 | CATTGTCTAACTTTAG | 31 | AAGAGT | H1 or |
| | | | | | GCAATR | | | H3/H2 |
| | | | Sa-KKH | 2A5 and 2G5 | TCTGCAGAGACTCTTY | 32 | TAAAGT | H1 or |
| | | | | | ATTGCC | | | H3/H2 |
| 3 | rs436527 | G/A | Sp-WT | 3R1 | ACGTGTGAGCC | 33 | GGG | H1, H2, H3 |
| | | | | | ACCACGCCC | | | |
| | | | Sp-WT | 3G2 | CACGTGTGAG | 34 | CGG | H1 |
| | | | | | CCACCACGCC | | | |
| | | | Sp-WT | 3G3 and 3A3 | TTAAATAAGAC | 35 | GGG | H1 or |
| | | | | | TTTAGGCCY | | | H3/H2 |
| | | | Sp-WT | 3G4 and 3A4 | TAAGACTTTAG | 36 | TGG | H1 or |
| | | | | | GCCYGGGCG | | | H3/H2 |
| | | | Sp-WT | 3G5 and 3A5 | GACTTTAGGCCY | 37 | TGG | H1 or |
| | | | | | GGGCGTGG | | | H3/H2 |
| | | | Sa-KKH | 3G6 and 3A6 | TGTGAGCCACC | 38 | TAAAGT | H1 or |
| | | | | | ACGCCCRGGCC | | | H3/H2 |
| | | | Sa-KKH | 3G7 and 3A7 | TAAATAAGACTTT | 39 | CGTGGT | H1 or |
| | | | | | AGGCCYGGG | | | H3/H2 |
| 4 | rs428854 | G/A | Sp-WT | 4G1 and 4A1 | TTTCTGCTTTCATA | 40 | TGG | H1 or |
| | | | | | AARCTA | | | H3/H2 |
| | | | Sp-VRQR | 4G2 and 4A2 | TTCTGCTTTCATAA | 41 | GGAN | H1 or |
| | | | | | ARCTAT | | | H3/H2 |
| | | | Sp-VRQR | 4G3 and 4A3 | ATCCATAGYTTTAT | 42 | AGAN | H1 or |
| | | | | | GAAAGC | | | H3/H2 |
| | | | Sp-VRQR | 4G4 and 4A4 | AACAGGTATCCATA | 43 | TGAA | H1 or |
| | | | | | GYTTTA | | | H3/H2 |
| | | | Sa-WT | 4G5 and 4A5 | ATGTTTCTGCTTTC | 44 | ATGGAT | H1 or |
| | | | | | ATAAARCT | | | H3/H2 |
| | | | Sa-KKH | 4A6 | TACACATATCAAAACA | 45 | CATACT | H2 |
| | | | | | GGTATC | | | |
| 5 | rs424499 | T/C | Sp-WT | 5T1 and 5C1 | CAGGAGAATYGCTTGA | 46 | TGG | H1 or |
| | | | | | ACCC | | | H3/H2 |
| | | | Sp-WT | 5T2 and 5C2 | GAGAATYGCTTGAAC | 47 | AGG | H1 or |
| | | | | | CCTGG | | | H3/H2 |
| | | | Sp-VRQR | 5T3 and 5C3 | GCTGAAGCAGGAGA | 48 | TGAA | H1 or |
| | | | | | ATYGCT | | | H3/H2 |
| | | | Sp-VRQR | 5T4 and 5C4 | AGGAGAATYGCTTG | 49 | GGAG | H1 or |
| | | | | | AACCCT | | | H3/H2 |
| | | | Sp-VRQR | 5C5 | CTCCACCTCCAGGG | 50 | CGAT | H2 |
| | | | | | TTCAAG | | | |
| 6 | rs429668 | T/C | Sp-VRQR | 6C1 | TCCAAAGGAA | 51 | AGAA | H2 |
| | | | | | GAGGCAGCAA | | | |
| 7 | rs444772 | G/A | Sp-WT | 7G1 and 7A1 | AAAACCAGA | 52 | AGG | H1 or |
| | | | | | AAAAACRTAA | | | H3/H2 |
| | | | Sp-WT | 7G2 and 7A2 | AAAGCCAGAA | 53 | GGG | H1 or |
| | | | | | AAAACRTAAA | | | H3/H2 |
| | | | Sp-WT | 7G3 and 7A3 | TATCCCCTTT | 54 | TGG | H1 or |
| | | | | | AYGTTTTTTC | | | H3/H2 |
| | | | Sp-WT | 7G4 and 7A4 | AAGCCAGAAA | 55 | GGG | H1 or |
| | | | | | AAACRTAAAG | | | H3/H2 |
| | | | Sp-VRQR | 7G5 and 7A5 | AGCCAGAAAA | 56 | GGAT | H1 or |
| | | | | | AACRTAAAGG | | | H3/H2 |
| | | | Sa-WT | 7G6 and 7A6 | AAAAAGCCAGA | 57 | GGGGAT | H1 or |
| | | | | | AAAAACRTAAA | | | H3/H2 |
| | | | Sa-KKH | 7G7 and 7A7 | GCCAGAAAAAA | 58 | TAAGT | H1 or |
| | | | | | CRTAAAGGGGA | | | H3/H2 |
| 8 | rs446227 | G/A | Sp-WT | 8G1 and 8A1 | ATCATAAAGA | 59 | TGG | H1 or |
| | | | | | CTTGYTTTCC | | | H3/H2 |
| | | | Sp-VRQR | 8G2 and 8A2 | TCCAGGAAAR | 60 | TGAT | H1 or |
| | | | | | CAAGTCTTTA | | | H3/H2 |
| | | | Sa-KKH | 8G3 and 8A3 | GTGTTCCAGGA | 61 | TATGAT | H1 or |
| | | | | | AARCAAGTCTT | | | H3/H2 |
| 9 | rs414352 | T/C | Sp-WT | 9T1 and 9C1 | TAGTAGTTCA | 62 | AGG | H1 or |
| | | | | | YCTATGTTGC | | | H3/H2 |
| | | | Sp-VRQR | 9T2 and 9C2 | AGTAGTTCAY | 63 | GGAA | H1 or |
| | | | | | CTATGTTGCA | | | H3/H2 |
| | | | Sp-VRQR | 9T3 and 9C3 | TGGAATTCCT | 64 | TGAA | H1 or |
| | | | | | GCAACATAGR | | | H3/H2 |
| | | | Sp-VRQR | 9T4 and 9C4 | TGCAACATAG | 65 | AGAT | H1 or |
| | | | | | RTGAACTACT | | | H3/H2 |
| | | | Sa-KKH | 9T5 and 9C5 | TTCCTGCAACAT | 66 | CTAGAT | H1 or |
| | | | | | AGRTGAACTA | | | H3/H2 |
| | | | Sa-WT | 9T6 and 9C6 | TCTAGTAGTTC | 67 | AGGAAT | H1 or |
| | | | | | AYCTATGTTGC | | | H3/H2 |
| 10 | rs441800 | A/G | Sp-VRQR | 10A1 and 10G1 | GAACCTGGTA | 68 | TGAT | H1 or |
| | | | | | CAAAACARAA | | | H3/H2 |
| | | | Sp-VRQR | 10A2 and 10G2 | CCTGGTACAA | 69 | TGAT | H1 or |
| | | | | | AACARAATGA | | | H3/H2 |
| | | | Sa-WT | 10G3 | CATTGTAGAAC | 70 | CAGAAT | H1 or |
| | | | | | CTGGTACAAAA | | | H3/H2 |
| | | | Sa-KKH | 10A4 and 10G4 | TGTAGAACCTG | 71 | AATGAT | H1 or |
| | | | | | GTACAAAACAR | | | H3/H2 |
| | | | Sa-KKH | 10A5 and 10G5 | AGAACCTGGTA | 72 | GATGAT | H1 or |
| | | | | | CAAAACARAAT | | | H3/H2 |
| | | | Sa-KKH | 10A6 and 10G6 | GCTATCATCAT | 73 | CCAGGT | H1 or |
| | | | | | TYTGTTTTGTA | | | H3/H2 |
| | | | Sa-WT | 10A7 and 10G7 | GTACAAAACAR | 74 | CAGAAT | H1 or |
| | | | | | AATGATGATAG | | | H3/H2 |
| 11 | rs62514616 | G/A | Sp-WT | 11G1 and 11A1 | TCCCGAATAGC | 75 | AGG | H1/H3 |
| | | | | | TGGGAYTAC | | | |
| | | | Sp-WT | 11G2 and 11A2 | GGCCTGTART | 76 | CGG | H1/H3 |
| | | | | | CCCAGCTATT | | | |
| | | | Sp-WT | 11G3 and 11A3 | GCCTGTARTCC | 77 | GGG | H1/H3 |
| | | | | | CAGCTATTC | | | |
| | | | Sa-WT | 11A4 | CTGCTTCAGCA | 78 | TGGGAT | H1/H3 |
| | | | | | TCCCGAATAGC | | | |
| 12 | rs2293869 | A/T | Sp-WT | 12A1 and 12T1 | TAGATWATCT | 79 | CGG | H1/H3 |
| | | | | | CCTGTCAAAC | | | |
| | | | Sp-WT | 12A2 and 12T2 | AGGAGATWAT | 80 | AGG | H1/H3 |
| | | | | | CTATGTAAAG | | | |
| | | | Sp-VRQR | 12A3 and 12T3 | ACAGGAGATW | 81 | AGAG | H1/H3 |
| | | | | | ATCTATGTAA | | | |
| | | | Sa-KKH | 12A4 | GACTTATCTCC | 82 | GATAAT | H1 |
| | | | | | CTCTTTACATA | | | |
| 13 | rs61739567 | G/A | Sp-VRQR | 13G1 and 13A1 | TTTGAAGGAA | 83 | TGAA | H1/H3 |
| | | | | | AGGTTTTRTA | | | |
| | | | Sa-WT | 13G2 and 13A2 | AGATTTGAAGG | 84 | ATGAAT | H1/H3 |
| | | | | | AAAGGTTTTR | | | |
| 14 | * | | Sp-WT | 14N1 | TTTGAAGGAA | 85 | TGAA | H1, H2, H3 |
| | | | | | AGGTTTTGTA | | | |
| | | | Sp-WT | 14N2 | AGATTTGAAGG | 86 | ATGAAT | H1, H2, H3 |
| | | | | | AAAGGTTTTG | | | |
| 15 | * | | Sp-VRQR | 15N1 | TTTGAAGGAA | 87 | TGAA | H1, H2, H3 |
| | | | | | AGGTTTTGTA | | | |
| | | | Sp-VRQR | 15N2 | AGATTTGAAGG | 88 | ATGAAT | H1, H2, H3 |
| | | | | | AAAGGTTTTG | | | |

*intronic region; not a SNP; R is A or G; W is A or T; Y is C or T; #, Protospacer SEQ ID NO:

### Cloning of Cas9 and sgRNA Expression Constructs

All Cas9 expression plasmids were constructed into a CAG-Cas9-T2A-EGFP backbone modified from a pSQT817 vector (Addgene #53373). EGFP and Cas9 were co-expressed as a single protein before being separated by a self-cleaving T2A peptide; EGFP served as the marker for the targeted cells and for the downstream FACS sorting. The sgRNAs for wild-type and variant SpCas9 were cloned into the expression vector BPK1520 (Addgene #65777), and the sgRNAs for wild-type and variant SaCas9 were cloned into the BPK2660 vector (Addgene #70709).

### Characterization of Human Cell Lines

We sourced and genotyped several human cell-lines to serve as homozygous and heterozygous RP1 haplotypes to evaluate the targeting allele-specificity and efficiency of our sgRNAs. These include HEK293T (H2/H3), ARPE-19 (H1/H2), hTERT RPE-1 (H3/H3), Min38931 fibroblast (H2/H2 from RP1 patient), HL-60 (H1/H1), HT-1080 (H1/H2), C3A (H2/H3), Y79 (H1/H3) Weri-Rb1 (H1/H2) and the haploid human cell-line HAP1 (H1).

HEK293T cells were selected for the initial screening step, for their efficiency of transfection, and heterozygous H2/H3 haplotype, such that all sgRNAs could be tested in these cells (in the H3 positions, H2 SNPs are the same as H1, and vice versa. Thus the reference haplotype H1 has 13 targetable sites, H2 has 10 sites, and H3 has 3 sites).

To assess the efficiency and specificity of each gRNA, we co-transfected CAG-Cas9-T2A-EGFP and sgRNA plasmids into HEK293T cells via Lipofectamine 3000. Transfection of the Cas9-EGFP plasmid alone was included as a negative control. Cells were collected 2 days after transfection, followed by FACS sorting. Genomic DNA was isolated from the sorted EGFP positive cells, PCR amplified, followed by NGS deep sequencing.

### Measurement of On-Target Efficiency

NGS deep sequencing was utilized to quantitatively analyze the specific DSBs-NHEJ events in the genome, and to predict the cleavage specificity. Briefly, short PCR products spanning the targeted site were amplified and purified, and dual-indexed TruSeq 250 bp paired end sequencing was performed on an Illumina MiSeq Sequencer by MGH CCIB. Paired-end reads were mapped to the human genome reference and reads with an average quality score >30 were analyzed for indels that overlap the intended target nuclease binding site. Indel analyses were conducted using an in-house algorithm at the MGH CCIB.

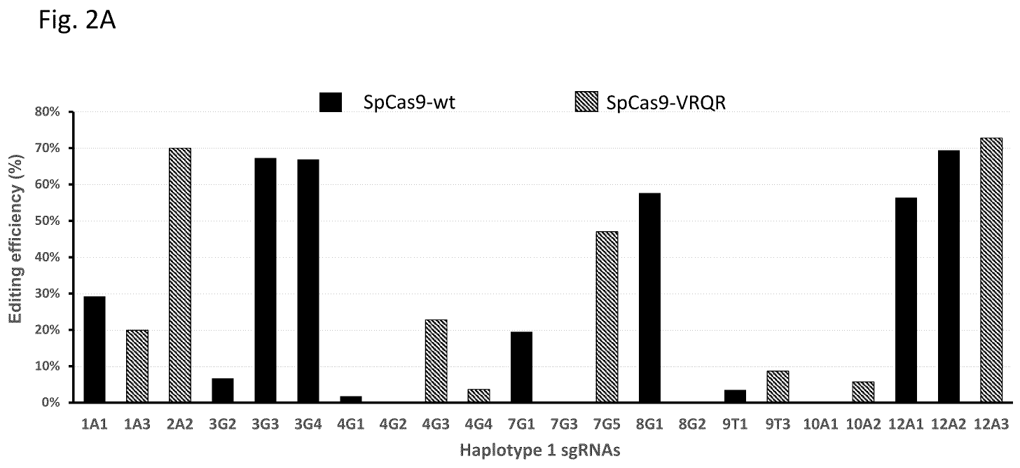

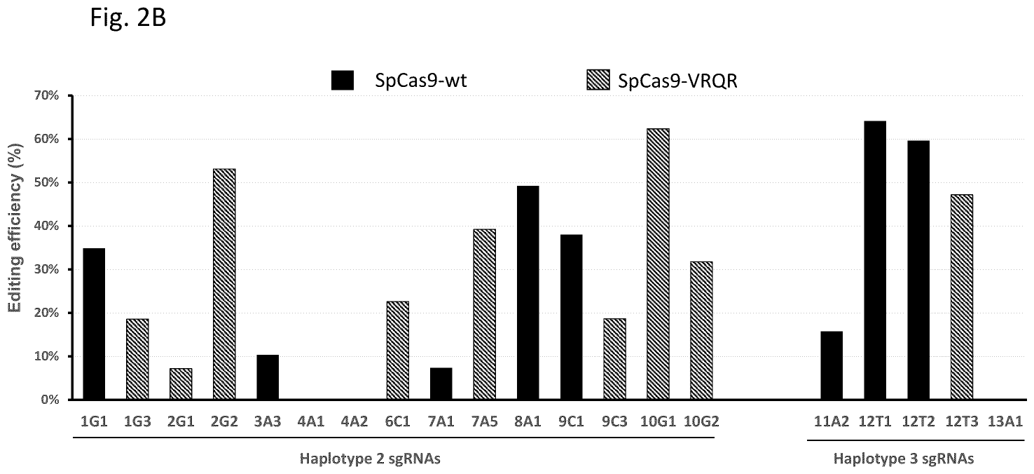

SpCas9 wild-type and variant guides for all three haplotypes have been tested in HEK293T, ARPE19, hTert-RPE1, U2OS, HAP1 and Weri-Rb1 cell lines, and analyzed for edited and unedited reads per allele. Editing efficiency was calculated as a ratio of edited alleles/total reads per allele, and are presented in Table 7 below, and FIGS. 2A and 2B.

TABLE 7
Indel rate of gRNAs targeting RP1 haplotypes

| Sites # | SNPs | SNP | RP1 | sgRNA ID | Editing on H1 | Editing on H2 | Editing on H3 |
|---|---|---|---|---|---|---|---|
| 1 | rs702761 | A > G | Intron 1 | 1A1 and 1G1 | 29.2% | 34.9% | |
| | | | | 1A2 and 1G2 | | | |
| | | | | 1A3 and 1G3 | 19.9% | 18.6% | |
| | | | | 1A4 and 1G4 | 0.0% | 0.0% | |
| | | | | 1A5 and 1G5 | 61.7% | 43.3% | |
| | | | | 1A6 and 1G6 | 0.0% | 3.5% | |
| 2 | rs145290 | A > G | Intron 1 | 2G1 | | 7.2% | |
| | | | | 2A2 and 2G2 | 70.0% | 53.1% | |
| | | | | 2A3 and 2G3 | | | |
| | | | | 2A4 and 2G4 | 0.0% | 0.0% | |
| | | | | 2A5 and 2G5 | 0.0% | 0.0% | |
| 3 | rs436527 | G > A | Intron 1 | 3G/A1 | | | |
| | | | | 3G2 | 6.7% | | |
| | | | | 3G3 and 3A3 | 67.3% | 10.4% | |
| | | | | 3G4 and 3A4 | 66.9% | | |
| | | | | 3G5 and 3A5 | | | |
| | | | | 3G6 and 3A6 | 24.2% | 28.9% | |
| | | | | 3G7 and 3A7 | 2.9% | 0.0% | |
| 4 | rs428854 | G > A | Intron 3 | 4G1 and 4A1 | 1.8% | 0.0% | |
| | | | | 4G2 and 4A2 | 0.0% | 0.0% | |
| | | | | 4G3 and 4A3 | 22.8% | | |
| | | | | 4G4 and 4A4 | 3.6% | | |
| | | | | 4G5 and 4A5 | 6.2% | 0.0% | |
| | | | | 4A6 | | | |
| 5 | rs424499 | T > C | Intron 3 | 5T1 and 5C1 | | | |
| | | | | 5T2 and 5C2 | | | |
| | | | | 5T3 and 5C3 | | | |
| | | | | 5T4 and 5C4 | | | |
| | | | | 5C5 | | | |
| 6 | rs429668 | T > C | Intron 3 | 6C1 | | 22.6% | |
| 7 | rs444772 | G > A | Exon 4 | 7G1 and 7A1 | 19.5% | 7.4% | |
| | | | | 7G2 and 7A2 | | | |
| | | | | 7G3 and 7A3 | 0.4% | | |
| | | | | 7G4 and 7A4 | | | |
| | | | | 7G5 and 7A5 | 47.0% | 39.2% | |
| | | | | 7G6 and 7A6 | | | |
| | | | | 7G7 and 7A7 | | | |
| 8 | rs446227 | G > A | Exon 4 | 8G1 and 8A1 | 57.7% | 49.2% | |
| | | | | 8G2 and 8A2 | | | |
| | | | | 8G3 and 8A3 | 0.0% | | |
| 9 | rs414352 | T > C | Exon 4 | 9T1 and 9C1 | 3.5% | 38.0% | |
| | | | | 9T2 and 9C2 | | | |
| | | | | 9T3 and 9C3 | 8.7% | 18.6% | |
| | | | | 9T4 and 9C4 | | | |
| | | | | 9T5 and 9C5 | | | |
| | | | | 9T6 and 9C6 | | | |
| 10 | rs441800 | A > G | Exon 4 | 10A1 and 10G1 | 0.0% | 62.4% | |
| | | | | 10A2 and 10G2 | 5.7% | 31.8% | |
| | | | | 10G3 | | | |
| | | | | 10A4 and 10G4 | | | |
| | | | | 10A5 and 10G5 | | | |
| | | | | 10A6 and 10G6 | | | |
| | | | | 10A7 and 10G7 | | | |
| 11 | rs62514616 | G > A | Intron 3 | 11G1 and 11A1 | | | |
| | | | | 11G2 and 11A2 | | | 15.7% |
| | | | | 11G3 and 11A3 | | | |
| | | | | 11A4 | | | |
| 12 | rs2293869 | A > T | Exon 4 | 12A1 and 12T1 | 56.4% | | 64.1% |
| | | | | 12A2 and 12T2 | 69.4% | | 59.7% |
| | | | | 12A3 and 12T3 | 72.8% | | 47.2% |
| | | | | 12A4 | | | |
| 13 | rs61739567 | G > A | Exon 4 | 13G1 and 13A1 | | | 0.0% |
| | | | | 13G2 and 13A2 | | | |
| 14 | wt | — | Intron 3 | 14N1 | 95.2% | | |
| | | — | | 14N2 | 88.7% | | |
| 15 | wt | — | Intron 3 | 15N1 | 51.6% | | |
| | | — | | 15N2 | 35.0% | | |

### Specific Knockout of RP1 Alleles by Cas9-sgRNA

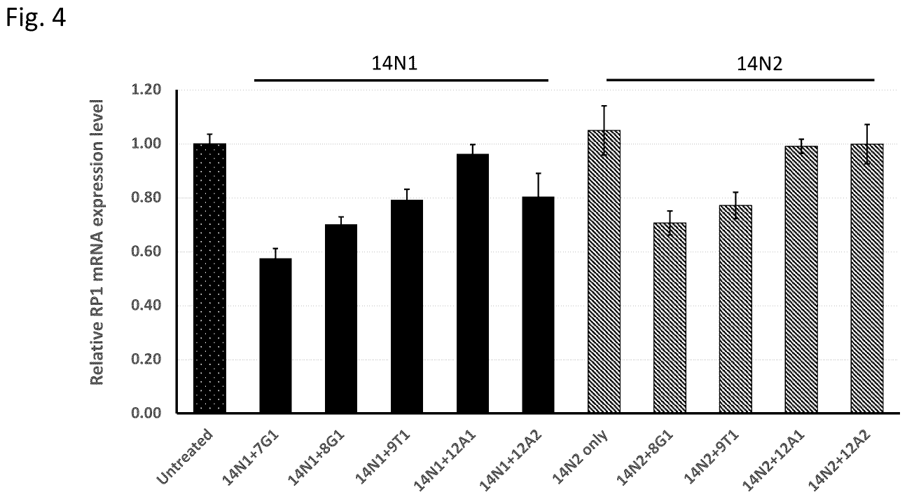

sgRNAs with the highest targeting efficiency and specificity were selected, and their ability to target and knockout RP1 alleles via paired sgRNA directed editing in heterozygous or haploid cell lines was evaluated. Paired guide deletion strategies for H1 were first tested. Editing by a single gRNA targeted to an intronic region was repaired by NHEJ, and did not affect RP1 expression (FIG. 4, single guide 14N2 only), therefore highly efficient guides targeting regions of intron 3 in any allele were paired with allele-specific sgRNAs targeting H1 SNPs in intron 1, or exon 4. Note that the bi-allele targeting guides will edit both wild type and mutant alleles, but when paired with allele specific guides in intron 1, or exon 4, will delete a large genomic portion form the mutant allele only. Larger deletions are also possible via the pairing of allele specific guides in intron 1 and exon 4 (Table 8 and Table 9).

TABLE 8
Possible combinations of sgRNAs to target
H1/H2, H3/H2 heterozygous

| Intron 1 | Intron 3 | Exon 4 |
|---|---|---|
| 1A1 and 1G1 | 14N1 | 10A2 and 10G2 |
| 1A5 and 1G5 | 14N2 | 7G1 and 7A1 |
| 2A2 and 2G2 | 15N1 | 7G5 and 7A5 |
| 3G3 and 3A3 | 15N2 | 8G1 and 8A1 |
| 3G4 and 3A4 | 4G3 | 9T1 and 9C1 |
| 3G6 and 3A6 | 5C5 | 9T3 and 9C3 |

TABLE 9
Possible combinations of sgRNAs
to target H1/H3 heterozygous

| Intron 3 | Exon 4 |
|---|---|
| 14N1 | 12A1 and 12T1 |
| 14N2 | 12A2 and 12T2 |
| 15N1 | 12A3 and 12T3 |
| 15N2 | |

### Quantification of Genomic Editing-qPCR Strategy

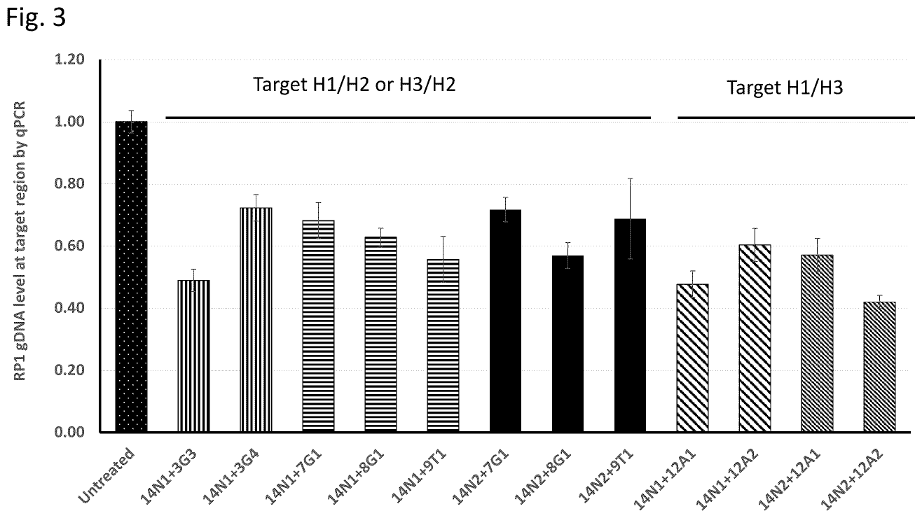

A qPCR strategy was developed to quantify editing and RP1 allele deletion efficiency at the genomic level. qPCR primers were designed to amplify regions within the predicted deletion. The ‘internal’ amplicon was produced only from remaining ‘undeleted’ alleles, and after normalization to a reference gene on another chromosome, the remaining ‘undeleted’ allele was quantified relative to unedited cells (FIG. 3). All combinations for sgRNAs tested were able to decrease RP1 allele content in edited cells, up to 58% for 14N2+12A2 gRNA pair. However, this quantification strategy may not capture all editing events. For example, inversions after dual DSBs may still allow amplification of ‘internal’ amplicons, if they have not been damaged by editing. Quantification of a reduction of RP1 expression is a more accurate measure of allele disruption, as described below.

### Quantification of RP1 Expression After Editing With Dual sgRNAs

Dual sgRNAs were transfected into the RP1 expressing cell-line Weri-Rb1. RNA was extracted from the GFP+ cell fraction with RNeasy Micro Plus (Qiagen), and cDNA libraries were generated for each sample with Superscript III First-Strand Synthesis SuperMix for qRT-PCR (Invitrogen). The sequences of cDNA specific primers for RP1, and the reference gene GAPDH, were obtained from OriGene, and RP1 expression was quantified in edited samples relative to non-transfected control cells (FIG. 4). A reduction in RP1 expression of 43% was observed in cells edited with 14N1+7G1 gRNAs. No change in RP1 expression was observed when the universal intron3 targeting guide 14N2 was individually tested.

### Generation of RP1 Expressing Cell-Lines

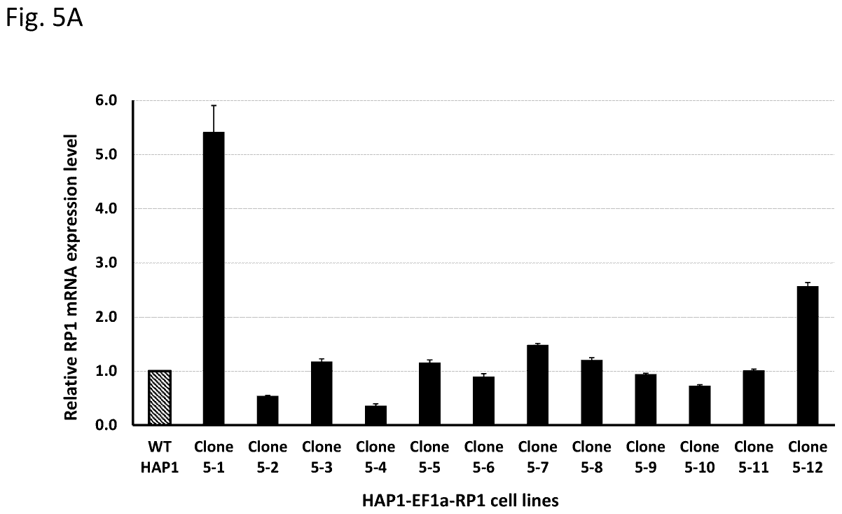

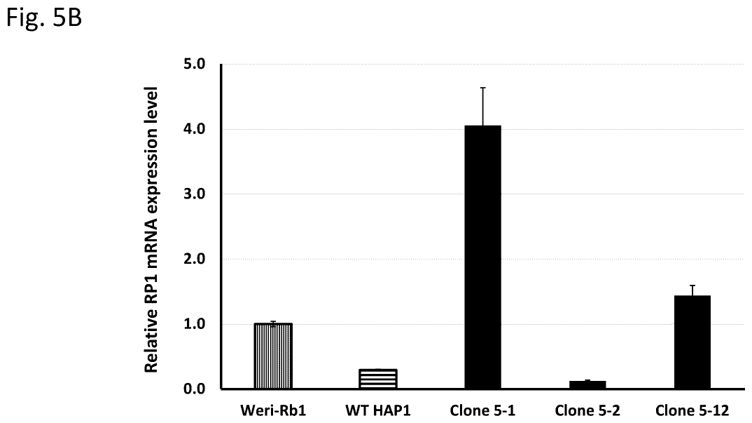

There are few commercially available cell lines in which to test the effect of our editing strategy on RP1 expression, since RP1 is specifically expressed in specialized post-mitotic photoreceptors. The retinoblastoma cell line Weri-Rb1 expresses low levels of RP1, but is difficult to transfect. Cell-lines were therefore generated with exogenous expression of RP1, by insertion of a promoter upstream of the RP1 transcriptional start site (TSS). A plasmid encoding 100 bp homology arms and the EF1a promoter followed by a neomycin resistance gene and a P2A peptide cleavage motif, was co-transfected with plasmids which express an RP1 targeting sgRNA, and SpCas9, into wild-type Hap1 cells. After 3 weeks of G418 selection at 50 ug/mL, 15 individual colonies were picked and analyzed for targeted genomic integration of the promoter cassette, and for RP1 expression by semi-quantitative RT-PCR. Clone 5 was then selected for stable cell line generation from single cells, and 12 clones were tested for relative RP1 expression by qRT-PCR (FIG. 5A). Three cell lines were then quantified for RP1 expression relative to the endogenously expressing cell line Weri-Rb1 (FIG. 5B). Clone 5-1 exhibited a four-fold increase in RP1 expression compared to Weri-Rb1, and is used to evaluate editing efficiency of the pairs of sgRNAs, and their effect on RP1 expression.

iPSCs have also been generated from fibroblasts obtained from two RP1 patients (OGI1557-002771 and OGI1781-003109) with dominant mutations selected from the probands in the IRD Biobank at MEEI. Patient OGI1557-002771 is heterozygous for H1/H3 haplotype, and carries the c. 2103delAATA mutation in cis with the SNPs for H3. Patient OGI1781-003109 is heterozygous for H2/H3, and carries the mutation c.2029C>T in cis with the SNPs for H2. Cells have undergone reprogramming with the non-integrating Sendaii virus method at Harvard iPSC Core Facility. These iPS cells and the differentiated retinal organoids are used to evaluate the lead Cas9/dual sgRNA pairs regarding their efficacy and safety.

Table 10 presents a summary of the SNPs and their presence in each haplotype, with the genomic information included.

TABLE 10
RP1 Haplotype SNPs with Genomic Location.

| | RP1 Haplotype variant | | | Genome |
|---|---|---|---|---|
| SNP | H1 | H2 | H3 | location |
| rs702761 | A | G | A | Intron 1 |
| rs145290 | A | G | A | Intron 1 |
| rs436527 | G | A | G | Intron 1 |
| rs428854 | G | A | G | Intron 3 |
| rs424499 | T | C | T | Intron 3 |
| rs429668 | T | C | T | Intron 3 |
| rs62514616 | G | G | A | Intron 3 |
| rs444772 | G | A | G | Exon 4 |
| rs446227 | G | A | G | Exon 4 |
| rs414352 | T | C | T | Exon 4 |
| rs441800 | A | G | A | Exon 4 |
| rs2293869 | A | A | T | Exon 4 |
| rs61739567 | G | G | A | Exon 4 |

Table 11 presents a summary of first and second gRNA targets by haplotype.

TABLE 11
First and Second gRNA Targets by Haplotype.

| Human | | | |
|---|---|---|---|
| subject | RP1 | | |
| RP1 | mutant | | |
| haplotype | allele | First gRNA target | Second gRNA target |
| H1/H2 | H1 | rs702761A, or rs145290A, or | rs428854G, or rs424499T, or rs429668T, or |
| | | rs436527G | rs444772G, or rs446227G, or rs414352T, |
| | | | or rs441800A |
| | | rs428854G, or rs424499T, or | rs444772G, or rs446227G, or rs414352T, |
| | | rs429668T | or rs441800A |
| | H2 | rs702761G, or rs145290G, or | rs428854A, or rs424499C, or rs429668C, or |
| | | rs436527A | rs444772A, or rs446227A, or rs414352C, or |
| | | | rs441800G |
| | | rs428854A, or rs424499C, or | rs444772A, or rs446227A, or rs414352C, or |
| | | rs429668C | rs441800G |
| H2/H3 | H2 | rs702761G, or rs145290G, or | rs428854A, or rs424499C, or rs429668C, or |
| | | rs436527A | rs62514616A, or rs444772A, or rs446227A, |
| | | | or rs414352C, or rs441800G, or |
| | | | rs2293869T, or rs61739567A |
| | | rs428854A, or rs424499C, or | rs444772A, or rs446227A, or rs414352C, or |
| | | rs429668C, or rs62514616A | rs441800G, or rs2293869T, or |
| | | | rs61739567A |
| | H3 | rs702761A, or rs145290A, or | rs428854G, or rs424499T, or rs429668T, or |
| | | rs436527G | rs62514616G, or rs444772G, or |
| | | | rs446227G, or rs414352T, or rs441800A, or |
| | | | rs2293869A, or rs61739567G |
| | | rs428854G, or rs424499T, or | rs444772G, or rs446227G, or rs414352T, |
| | | rs429668T, or rs62514616G | or rs441800A, or rs2293869A, or |
| | | | rs61739567G |
| H1/H3 | H1 | rs62514616G | rs2293869A, or rs61739567G |
| H1/H3 | H3 | rs62514616A | rs2293869T, or rs61739567A |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-17. (canceled)

18. A method of altering the genome of a cell, the method comprising using CRISPR editing to form a first double strand break within intron 1 or 3 of the human RP1 gene and a second double strand within intron 3 or 4 of the human RP1 gene.

19. The method of claim 18, wherein the cell is a cell of the eye of a mammal.

20. The method of claim 18, wherein the first and second double strand breaks are generated using:
(i) a RNA-guided nuclease (RGN) or a variant thereof, and
(ii) a first guide RNA (gRNA) and a second gRNA, wherein the first and second gRNAs target haplotype variants on the same allele of an human RP1 gene, and wherein the first and second gRNAs both target linked variants in haplotype H1, or H2, or H3, wherein:
(a) the first gRNA is targeted to a single nucleotide polymorphism (SNP) in intron 1 of an RP1 gene of a human subject selected from rs702761, rs145290, and rs436527, and the second gRNA is targeted to a SNP selected from rs428854, rs424499, and rs429668 in intron 3, or a SNP selected from rs444772, rs446227, rs414352, rs441800, rs2293869, and rs61739567 in exon 4 of the same haplotype of an human RP1 gene, or
(b) the first gRNA is targeted to a SNP selected from rs428854, rs424499, rs429668, and rs62514616 in intron 3 of an RP1 gene of a human subject; and the second gRNA is targeted to a SNP selected from rs444772, rs446227, rs414352, rs441800, rs2293869, and rs61739567 in exon 4 of the same haplotype of an human RP1 gene,
and wherein the haplotypes H1, H2, and H3 are:

| | RP1 Haplotype variant | | | Genome |
|---|---|---|---|---|
| SNP | H1 | H2 | H3 | location |
| rs702761 | A | G | A | Intron 1 |
| rs145290 | A | G | A | Intron 1 |
| rs436527 | G | A | G | Intron 1 |
| rs428854 | G | A | G | Intron 3 |
| rs424499 | T | C | T | Intron 3 |
| rs429668 | T | C | T | Intron 3 |
| rs62514616 | G | G | A | Intron 3 |
| rs444772 | G | A | G | Exon 4 |
| rs446227 | G | A | G | Exon 4 |
| rs414352 | T | C | T | Exon 4 |
| rs441800 | A | G | A | Exon 4 |
| rs2293869 | A | A | T | Exon 4 |
| rs61739567 | G | G | A | Exon 4. |

21. (canceled)

22. The method of claim 20, wherein the first and/or second gRNA comprise a protospacer sequence of one of SEQ ID NOs: 22-88.

23. The method of claim 18, wherein the cell is from a subject suffering from autosomal dominant retinitis pigmentosa (adRP).

24. The method of claim 18, wherein the cell is a retinal cell or a photoreceptor cell.

25. The method of claim 24, wherein the photoreceptor cell is a cone photoreceptor cell or a cone cell, a rod photoreceptor cell or a rod cell or a macular cone photoreceptor cell.

26. A method of altering a cell, comprising contacting the cell with a recombinant viral particle comprising:
a nucleotide sequence encoding a first gRNA comprising a sequence targeting a domain in intron 1 or 3 of the human RP1 gene, preferably as shown in Table 6;
a nucleotide sequence encoding a second gRNA molecule comprising a sequence targeting a domain in intron 3 or exon 4 of the human RP1 gene, preferably as shown in Table 6; and
a nucleotide sequence encoding a Cas9 molecule;
wherein said viral particle is capable of delivery to a non-dividing cell, and wherein said contacting results in removal of a) all of exon 2 and exon 3; (b) part of exon 4; or (c) all of exon 2 and exon 3 and part of exon 4; of a mutant allele of an RP1 gene on chromosome 8, sufficient to disrupt expression of the RP1 protein from that allele.

27. The method of claim 26, wherein the viral particle is an adeno-associated virus (AAV) viral particle.

28. (canceled)

29. The method of claim 20, wherein the RGN is S. aureus Cas9 or S. pyogenes Cas9 or a variant thereof.

30. The method of claim 24, wherein the Cas9 comprises a nuclear localization signal, optionally a C-terminal nuclear localization signal and/or an N-terminal nuclear localization signal; and/or wherein the sequence encoding Cas9 comprises a polyadenylation signal.

31. The method of claim 20, wherein the Cas9 protein is S. aureus Cas9 or a variant thereof and the first and/or the second gRNA is a unimolecular S. aureus gRNA comprising SEQ ID NO: 1 or SEQ ID NO: 2, or the corresponding two-part modular S. aureus gRNA, wherein the crRNA component comprises SEQ ID NO: 3 or SEQ ID NO: 4 and the tracrRNA component comprises SEQ ID NO: 5 or SEQ ID NO: 6.

32. The method of claim 20, wherein the Cas9 protein is S. pyogenes Cas9, or a variant thereof, and the first and/or the second gRNA is an S. pyogenes gRNA comprising any one of the sequences set forth in SEQ ID NOs: 7-16.

33. The method of claim 20, wherein the RGN and/or guide RNAs are delivered to the cell one or more viral delivery vectors.

34. The method of claim 33, wherein the viral delivery vectors comprise a promoter operably linked to the RGN, wherein the promoter is selected from cytomegalovirus (CMV), chicken β-actin (CBA), cytomegalovirus (CMV) enhancer fused to the chicken beta-actin promoter (CAG), CBh, elongation factor alpha 1 (EFalpha1), EF-1 Alpha Short (EFS), CASI, RHO, beta phosphodiesterase (PDE), retinitis pigmentosa (RP1), rhodopsin kinase (hGRK1), or cone arrestin (CAR) promoter.

35. The method of claim 33, wherein the viral delivery vectors are adeno-associated virus (AAV) vectors.

36. The method of claim 33, wherein the viral delivery vectors comprise a first and a second inverted terminal repeat sequence (ITR) and;
(i) sequences encoding one or both of the first guide RNA (gRNA) and the second gRNA, and
a U6 promoter for driving expression of the first and/or second gRNAs; and/or
(ii) a sequence encoding the RGN or variant thereof, and a promoter operably linked to the RGN.

37. A method of treating a subject who has a condition associated with a mutation in RP1, the method comprising administering to the eye of the subject a therapeutically effective amount of a composition comprising:
(i) a RNA-guided nuclease (RGN) or a variant thereof, and
(ii) a first guide RNA (gRNA) and a second gRNA, wherein the first and second gRNAs target haplotype variants on the same allele of an human RP1 gene, and wherein the first and second gRNAs both target linked variants in haplotype H1, or H2, or H3, wherein:
(a) the first gRNA is targeted to a single nucleotide polymorphism (SNP) in intron 1 of an RP1 gene of a human subject selected from rs702761, rs145290, and rs436527, and the second gRNA is targeted to a SNP selected from rs428854, rs424499, and rs429668 in intron 3, or a SNP selected from rs444772, rs446227, rs414352, rs441800, rs2293869, and rs61739567 in exon 4 of the same haplotype of an human RP1 gene, or
(b) the first gRNA is targeted to a SNP selected from rs428854, rs424499, rs429668, and rs62514616 in intron 3 of an RP1 gene of a human subject; and the second gRNA is targeted to a SNP selected from rs444772, rs446227, rs414352, rs441800, rs2293869, and rs61739567 in exon 4 of the same haplotype of an human RP1 gene,
and wherein the haplotypes H1, H2, and H3 are:

| | RP1 Haplotype variant | | | Genome |
|---|---|---|---|---|
| SNP | H1 | H2 | H3 | location |
| rs702761 | A | G | A | Intron 1 |
| rs145290 | A | G | A | Intron 1 |
| rs436527 | G | A | G | Intron 1 |
| rs428854 | G | A | G | Intron 3 |
| rs424499 | T | C | T | Intron 3 |
| rs429668 | T | C | T | Intron 3 |
| rs62514616 | G | G | A | Intron 3 |
| rs444772 | G | A | G | Exon 4 |
| rs446227 | G | A | G | Exon 4 |
| rs414352 | T | C | T | Exon 4 |
| rs441800 | A | G | A | Exon 4 |
| rs2293869 | A | A | T | Exon 4 |
| rs61739567 | G | G | A | Exon 4. |

38. The method of claim 37, wherein the condition is autosomal dominant retinitis pigmentosa (adRP).


SEQUENCE LISTING (88 sequences)

SEQ ID NO: 1 - DNA, 102 bp, Staphylococcus aureus
  variation 1..20: note = Targeting domain of 16-24 nts, each n=A, C, T, G, unknown or other
  source 1..102: mol_type = genomic DNA
nnnnnnnnnnnnnnnnnnnngttttagtactctggaaacagaatctactaaaacaaggcaaaatgccgtgtttatctcgtcaacttgttggcgagatttttt

SEQ ID NO: 2 - DNA, 102 bp, Staphylococcus aureus
  variation 1..20: note = Targeting domain of 16-24 nts, each n=A, C, T, G, unknown or other
  source 1..102: mol_type = genomic DNA
nnnnnnnnnnnnnnnnnnnngttatagtactctggaaacagaatctactataacaaggcaaaatgccgtgtttatctcgtcaacttgttggcgagatttttt

SEQ ID NO: 3 - DNA, 14 bp, Staphylococcus aureus
  source 1..14: mol_type = genomic DNA
gttttagtactctg

SEQ ID NO: 4 - DNA, 14 bp, Staphylococcus aureus
  source 1..14: mol_type = genomic DNA
gttatagtactctg

SEQ ID NO: 5 - DNA, 64 bp, Staphylococcus aureus
  source 1..64: mol_type = genomic DNA
cagaatctactaaaacaaggcaaaatgccgtgtttatctcgtcaacttgttggcgagatttttt

SEQ ID NO: 6 - DNA, 64 bp, Staphylococcus aureus
  source 1..64: mol_type = genomic DNA
cagaatctactataacaaggcaaaatgccgtgtttatctcgtcaacttgttggcgagatttttt

SEQ ID NO: 7 - RNA, 32 nt, Streptococcus pyogenes
  variation 1..20: note = n is A, C, U, G, unknown, or other and this region may encompass 17-20 nucleotides
  source 1..32: mol_type = genomic RNA
nnnnnnnnnnnnnnnnnnnngttttagagcta

SEQ ID NO: 8 - RNA, 42 nt, Streptococcus pyogenes
  variation 1..20: note = n is A, C, U, G, unknown, or other and this region may encompass 17-20 nucleotides
  source 1..42: mol_type = genomic RNA
nnnnnnnnnnnnnnnnnnnngttttagagctatgctgttttg

SEQ ID NO: 9 - RNA, 36 nt, Streptococcus pyogenes
  variation 1..20: note = N is A, C, U, G, unknown, or other and this region may encompass 17-20 nucleotides
  source 1..36: mol_type = genomic RNA
nnnnnnnnnnnnnnnnnnnngttttagagctatgct

SEQ ID NO: 10 - RNA, 62 nt, Streptococcus pyogenes
  variation 1..20: note = N is A, C, U, G, unknown, or other and this region may encompass 17-20 nucleotides
  source 1..62: mol_type = genomic RNA
nnnnnnnnnnnnnnnnnnnngttttagagctagaaatagcaagttaaaataaggctagtccg

SEQ ID NO: 11 - RNA, 75 nt, Streptococcus pyogenes
  variation 1..20: note = N is A, C, U, G, unknown, or other and this region may encompass 17-20 nucleotides
  source 1..75: mol_type = genomic RNA
nnnnnnnnnnnnnnnnnnnngttttagagctatgctgaaaagcatagcaagttaaaataaggctagtccgttatc

SEQ ID NO: 12 - RNA, 87 nt, Streptococcus pyogenes
  variation 1..20: note = N is A, C, U, G, unknown, or other and this region may encompass 17-20 nucleotides
  source 1..87: mol_type = genomic RNA
nnnnnnnnnnnnnnnnnnnngttttagagctatgctgttttggaaacaaaacagcatagcaagttaaaataaggctagtccgttatc

SEQ ID NO: 13 - RNA, 96 nt, Streptococcus pyogenes
  variation 1..20: note = N is A, C, U, G, unknown, or other and this region may encompass 17-20 nucleotides
  source 1..96: mol_type = genomic RNA
nnnnnnnnnnnnnnnnnnnngttttagagctagaaatagcaagttaaaataaggctagtccgttatcaacttgaaaaagtggcaccgagtcggtgc

SEQ ID NO: 14 - RNA, 96 nt, Streptococcus pyogenes
  variation 1..20: note = N is A, C, U, G, unknown, or other and this region may encompass 17-20 nucleotides
  source 1..96: mol_type = genomic RNA
nnnnnnnnnnnnnnnnnnnngtttaagagctagaaatagcaagtttaaataaggctagtccgttatcaacttgaaaaagtggcaccgagtcggtgc

SEQ ID NO: 15 - RNA, 106 nt, Streptococcus pyogenes
  variation 1..20: note = N is A, C, U, G, unknown, or other and this region may encompass 17-20 nucleotides
  source 1..106: mol_type = genomic RNA
nnnnnnnnnnnnnnnnnnnngttttagagctatgctggaaacagcatagcaagtttaaataaggctagtccgttatcaacttgaaaaagtggcaccgagtcggtgc

SEQ ID NO: 16 - RNA, 106 nt, Streptococcus pyogenes
  variation 1..20: note = N is A, C, U, G, unknown, or other and this region may encompass 17-20 nucleotides
  source 1..106: mol_type = genomic RNA
nnnnnnnnnnnnnnnnnnnngtttaagagctatgctggaaacagcatagcaagtttaaataaggctagtccgttatcaacttgaaaaagtggcaccgagtcggtgc

SEQ ID NO: 17 - protein, 7 aa, synthetic construct
PKKKRRV

SEQ ID NO: 18 - protein, 7 aa, synthetic construct
PKKKRKV

SEQ ID NO: 19 - protein, 18 aa, synthetic construct
KRTADGSEFESPKKKRKV

SEQ ID NO: 20 - protein, 16 aa, synthetic construct
KRPAATKKAGQAKKKK

SEQ ID NO: 21 - DNA, 60 bp, synthetic construct
  misc_feature 1..60: note = polyadenylation signal
  source 1..60: mol_type = other DNA
tagcaataaaggatcgtttattttcattggaagcgtgtgttggttttttgatcaggcgcg

SEQ ID NO: 22 - DNA, 20 bp, synthetic construct
  misc_feature 1..20: note = Protospacer
  source 1..20: mol_type = other DNA
atgctgatattgaartagaa

SEQ ID NO: 23 - DNA, 20 bp, synthetic construct
  misc_feature 1..20: note = Protospacer
  source 1..20: mol_type = other DNA
tgctgatattgaartagaat

SEQ ID NO: 24 - DNA, 20 bp, synthetic construct
  misc_feature 1..20: note = Protospacer
  source 1..20: mol_type = other DNA
caatatgctgatattgaart

SEQ ID NO: 25 - DNA, 22 bp, synthetic construct
  misc_feature 1..22: note = Protospacer
  source 1..22: mol_type = other DNA
tcacaatatgctgatattgaar

SEQ ID NO: 26 - DNA, 22 bp, synthetic construct
  misc_feature 1..22: note = Protospacer
  source 1..22: mol_type = other DNA
tgatattgaartagaatggaac

SEQ ID NO: 27 - DNA, 22 bp, synthetic construct
  misc_feature 1..22: note = Protospacer
  source 1..22: mol_type = other DNA
tgatactgaagttccattctay

SEQ ID NO: 28 - DNA, 20 bp, synthetic construct
  misc_feature 1..20: note = Protospacer
  source 1..20: mol_type = other DNA
cattgtctaactttaggcaa

SEQ ID NO: 29 - DNA, 20 bp, synthetic construct
  misc_feature 1..20: note = Protospacer
  source 1..20: mol_type = other DNA
tgtctaactttaggcaatra

SEQ ID NO: 30 - DNA, 20 bp, synthetic construct
  misc_feature 1..20: note = Protospacer
  source 1..20: mol_type = other DNA
aggcaatraagagtctctgc

SEQ ID NO: 31 - DNA, 22 bp, synthetic construct
  misc_feature 1..22: note = Protospacer
  source 1..22: mol_type = other DNA
cattgtctaactttaggcaatr

SEQ ID NO: 32 - DNA, 22 bp, synthetic construct
  misc_feature 1..22: note = Protospacer
  source 1..22: mol_type = other DNA
tctgcagagactcttyattgcc

SEQ ID NO: 33 - DNA, 20 bp, synthetic construct
  misc_feature 1..20: note = Protospacer
  source 1..20: mol_type = other DNA
acgtgtgagccaccacgccc

SEQ ID NO: 34 - DNA, 20 bp, synthetic construct
  misc_feature 1..20: note = Protospacer
  source 1..20: mol_type = other DNA
cacgtgtgagccaccacgcc

SEQ ID NO: 35 - DNA, 20 bp, synthetic construct
  misc_feature 1..20: note = Protospacer
  source 1..20: mol_type = other DNA
ttaaataagactttaggccy

SEQ ID NO: 36 - DNA, 20 bp, synthetic construct
  misc_feature 1..20: note = Protospacer
  source 1..20: mol_type = other DNA
taagactttaggccygggcg

SEQ ID NO: 37 - DNA, 20 bp, synthetic construct
  misc_feature 1..20: note = Protospacer
  source 1..20: mol_type = other DNA
gactttaggccygggcgtgg

SEQ ID NO: 38 - DNA, 22 bp, synthetic construct
  misc_feature 1..22: note = Protospacer
  source 1..22: mol_type = other DNA
tgtgagccaccacgcccrggcc

SEQ ID NO: 39 - DNA, 22 bp, synthetic construct
  misc_feature 1..22: note = Protospacer
  source 1..22: mol_type = other DNA
taaataagactttaggccyggg

SEQ ID NO: 40 - DNA, 20 bp, synthetic construct
  misc_feature 1..20: note = Protospacer
  source 1..20: mol_type = other DNA
tttctgctttcataaarcta

SEQ ID NO: 41 - DNA, 20 bp, synthetic construct
  misc_feature 1..20: note = Protospacer
  source 1..20: mol_type = other DNA
ttctgctttcataaarctat

SEQ ID NO: 42 - DNA, 20 bp, synthetic construct
  misc_feature 1..20: note = Protospacer
  source 1..20: mol_type = other DNA
atccatagytttatgaaagc

SEQ ID NO: 43 - DNA, 20 bp, synthetic construct
  misc_feature 1..20: note = Protospacer
  source 1..20: mol_type = other DNA
aacaggtatccatagyttta

SEQ ID NO: 44 - DNA, 22 bp, synthetic construct
  misc_feature 1..22: note = Protospacer
  source 1..22: mol_type = other DNA
atgtttctgctttcataaarct

SEQ ID NO: 45 - DNA, 22 bp, synthetic construct
  misc_feature 1..22: note = Protospacer
  source 1..22: mol_type = other DNA
tacacatatcaaaacaggtatc

SEQ ID NO: 46 - DNA, 20 bp, synthetic construct
  misc_feature 1..20: note = Protospacer
  source 1..20: mol_type = other DNA
caggagaatygcttgaaccc

SEQ ID NO: 47 - DNA, 20 bp, synthetic construct
  misc_feature 1..20: note = Protospacer
  source 1..20: mol_type = other DNA
gagaatygcttgaaccctgg

SEQ ID NO: 48 - DNA, 20 bp, synthetic construct
  misc_feature 1..20: note = Protospacer
  source 1..20: mol_type = other DNA
gctgaagcaggagaatygct

SEQ ID NO: 49 - DNA, 20 bp, synthetic construct
  misc_feature 1..20: note = Protospacer
  source 1..20: mol_type = other DNA
aggagaatygcttgaaccct

SEQ ID NO: 50 - DNA, 20 bp, synthetic construct
  misc_feature 1..20: note = Protospacer
  source 1..20: mol_type = other DNA
ctccacctccagggttcaag

SEQ ID NO: 51 - DNA, 20 bp, synthetic construct
  misc_feature 1..20: note = Protospacer
  source 1..20: mol_type = other DNA
tccaaaggaagaggcagcaa

SEQ ID NO: 52 - DNA, 20 bp, synthetic construct
  misc_feature 1..20: note = Protospacer
  source 1..20: mol_type = other DNA
aaaagccagaaaaaacrtaa

SEQ ID NO: 53 - DNA, 20 bp, synthetic construct
  misc_feature 1..20: note = Protospacer
  source 1..20: mol_type = other DNA
aaagccagaaaaaacrtaaa

SEQ ID NO: 54 - DNA, 20 bp, synthetic construct
  misc_feature 1..20: note = Protospacer
  source 1..20: mol_type = other DNA
tatcccctttaygttttttc

SEQ ID NO: 55 - DNA, 20 bp, synthetic construct
  misc_feature 1..20: note = Protospacer
  source 1..20: mol_type = other DNA
aagccagaaaaaacrtaaag

SEQ ID NO: 56 - DNA, 20 bp, synthetic construct
  misc_feature 1..20: note = Protospacer
  source 1..20: mol_type = other DNA
agccagaaaaaacrtaaagg

SEQ ID NO: 57 - DNA, 22 bp, synthetic construct
  misc_feature 1..22: note = Protospacer
  source 1..22: mol_type = other DNA
aaaaagccagaaaaaacrtaaa

SEQ ID NO: 58 - DNA, 22 bp, synthetic construct
  misc_feature 1..22: note = Protospacer
  source 1..22: mol_type = other DNA
gccagaaaaaacrtaaagggga

SEQ ID NO: 59 - DNA, 20 bp, synthetic construct
  misc_feature 1..20: note = Protospacer
  source 1..20: mol_type = other DNA
atcataaagacttgytttcc

SEQ ID NO: 60 - DNA, 20 bp, synthetic construct
  misc_feature 1..20: note = Protospacer
  source 1..20: mol_type = other DNA
tccaggaaarcaagtcttta

SEQ ID NO: 61 - DNA, 22 bp, synthetic construct
  misc_feature 1..22: note = Protospacer
  source 1..22: mol_type = other DNA
gtgttccaggaaarcaagtctt

SEQ ID NO: 62 - DNA, 20 bp, synthetic construct
  misc_feature 1..20: note = Protospacer
  source 1..20: mol_type = other DNA
tagtagttcayctatgttgc

SEQ ID NO: 63 - DNA, 20 bp, synthetic construct
  misc_feature 1..20: note = Protospacer
  source 1..20: mol_type = other DNA
agtagttcayctatgttgca

SEQ ID NO: 64 - DNA, 20 bp, synthetic construct
  misc_feature 1..20: note = Protospacer
  source 1..20: mol_type = other DNA
tggaattcctgcaacatagr

SEQ ID NO: 65 - DNA, 20 bp, synthetic construct
  misc_feature 1..20: note = Protospacer
  source 1..20: mol_type = other DNA
tgcaacatagrtgaactact

SEQ ID NO: 66 - DNA, 22 bp, synthetic construct
  misc_feature 1..22: note = Protospacer
  source 1..22: mol_type = other DNA
ttcctgcaacatagrtgaacta

SEQ ID NO: 67 - DNA, 22 bp, synthetic construct
  misc_feature 1..22: note = Protospacer
  source 1..22: mol_type = other DNA
tctagtagttcayctatgttgc

SEQ ID NO: 68 - DNA, 20 bp, synthetic construct
  misc_feature 1..20: note = Protospacer
  source 1..20: mol_type = other DNA
gaacctggtacaaaacaraa

SEQ ID NO: 69 - DNA, 20 bp, synthetic construct
  misc_feature 1..20: note = Protospacer
  source 1..20: mol_type = other DNA
cctggtacaaaacaraatga

SEQ ID NO: 70 - DNA, 22 bp, synthetic construct
  misc_feature 1..22: note = Protospacer
  source 1..22: mol_type = other DNA
cattgtagaacctggtacaaaa

SEQ ID NO: 71 - DNA, 22 bp, synthetic construct
  misc_feature 1..22: note = Protospacer
  source 1..22: mol_type = other DNA
tgtagaacctggtacaaaacar

SEQ ID NO: 72 - DNA, 22 bp, synthetic construct
  misc_feature 1..22: note = Protospacer
  source 1..22: mol_type = other DNA
agaacctggtacaaaacaraat

SEQ ID NO: 73 - DNA, 22 bp, synthetic construct
  misc_feature 1..22: note = Protospacer
  source 1..22: mol_type = other DNA
gctatcatcattytgttttgta

SEQ ID NO: 74 - DNA, 22 bp, synthetic construct
  misc_feature 1..22: note = Protospacer
  source 1..22: mol_type = other DNA
gtacaaaacaraatgatgatag

SEQ ID NO: 75 - DNA, 20 bp, synthetic construct
  misc_feature 1..20: note = Protospacer
  source 1..20: mol_type = other DNA
tcccgaatagctgggaytac

SEQ ID NO: 76 - DNA, 20 bp, synthetic construct
  misc_feature 1..20: note = Protospacer
  source 1..20: mol_type = other DNA
ggcctgtartcccagctatt

SEQ ID NO: 77 - DNA, 20 bp, synthetic construct
  misc_feature 1..20: note = Protospacer
  source 1..20: mol_type = other DNA
gcctgtartcccagctattc

SEQ ID NO: 78 - DNA, 22 bp, synthetic construct
  misc_feature 1..22: note = Protospacer
  source 1..22: mol_type = other DNA
ctgcttcagcatcccgaatagc

SEQ ID NO: 79 - DNA, 20 bp, synthetic construct
  misc_feature 1..20: note = Protospacer
  source 1..20: mol_type = other DNA
tagatwatctcctgtcaaac

SEQ ID NO: 80 - DNA, 20 bp, synthetic construct
  misc_feature 1..20: note = Protospacer
  source 1..20: mol_type = other DNA
aggagatwatctatgtaaag

SEQ ID NO: 81 - DNA, 20 bp, synthetic construct
  misc_feature 1..20: note = Protospacer
  source 1..20: mol_type = other DNA
acaggagatwatctatgtaa

SEQ ID NO: 82 - DNA, 22 bp, synthetic construct
  misc_feature 1..22: note = Protospacer
  source 1..22: mol_type = other DNA
gacttatctccctctttacata

SEQ ID NO: 83 - DNA, 20 bp, synthetic construct
  misc_feature 1..20: note = Protospacer
  source 1..20: mol_type = other DNA
tttgaaggaaaggttttrta

SEQ ID NO: 84 - DNA, 21 bp, synthetic construct
  misc_feature 1..21: note = Protospacer
  source 1..21: mol_type = other DNA
agatttgaaggaaaggttttr

SEQ ID NO: 85 - DNA, 20 bp, synthetic construct
  misc_feature 1..20: note = Protospacer
  source 1..20: mol_type = other DNA
tttgaaggaaaggttttgta

SEQ ID NO: 86 - DNA, 21 bp, synthetic construct
  misc_feature 1..21: note = Protospacer
  source 1..21: mol_type = other DNA
agatttgaaggaaaggttttg

SEQ ID NO: 87 - DNA, 20 bp, synthetic construct
  misc_feature 1..20: note = Protospacer
  source 1..20: mol_type = other DNA
tttgaaggaaaggttttgta

SEQ ID NO: 88 - DNA, 21 bp, synthetic construct
  misc_feature 1..21: note = Protospacer
  source 1..21: mol_type = other DNA
agatttgaaggaaaggttttg